(19) United States
(12) Patent Application Publication
(10) Pub. No.: US 2003/0191049 A1
(43) Pub. Date: Oct. 9, 2003

(54) Oligomers of nonpeptide restricted mimetics of dipeptides of tripeptides, and the use thereof in the synthesis of synthetic proteins and polypeptides

(76) Inventors: Muriel Amblard, Saint Jean De Vedas (FR); Jean Martinez, Saussan (FR); Gilbert Berge, Montpellier (FR)

Correspondence Address: BURNS DOANE SWECKER & MATHIS L L P, POST OFFICE BOX 1404, ALEXANDRIA, VA, 22313-1404, US

(21) Appl. No.: 10/169,907

(22) PCT Filed: Jan. 11, 2001
(86) PCT No.: PCT/FR01/00088

(30) Foreign Application Priority Data
Jan. 11, 2000 (FR) 00/00288

(51) Int. Cl. C07K 7/02

(52) U.S. Cl. 514/2; 530/327; 530/328

(57) ABSTRACT

The invention relates to nonpeptide oligomers of amino acids. The oligomers comprise —(NR′-A-CO)—O— units which represent a nonpeptide, restricted-mimetic inducer of the β turn in a dipeptide or tripeptide fragment. Said oligomers may be produced by peptide synthesis techniques, whether in solution or in the solid phase, and can be used in the synthesis of synthetic proteins or polypeptides, in which the peptide fragment(s) (is) are identical to those of the corresponding natural protein or polypeptide and whose structural fragment(s) comprise(s) a fragment of an oligomer according to the invention.

The present invention relates to oligomers of amino acids, to a method for the preparation thereof, to the use thereof for synthesizing artificial polypeptides and proteins, and to the artificial polypeptides and proteins obtained.

Proteins and polypeptides are polymers obtained by coupling a certain number of identical or different α-amino acids in a given order. They have many highly advantageous properties, but their structure makes them fragile and they are easily degraded.

An attempt has been made to increase the stability by replacing all or some of the α-amino acids in the sequence of a natural polypeptide or protein with β-amino acids (K. Gademan et al., Helvetica Chimica Acta Vol. 82 (1999), pp. 1-11). This substitution effectively made it possible to improve the stability, activity and structuring of the products obtained. β-amino acids are not, however, constrained entities and are close to the structures of α-amino acids.

Polyamides obtained by polymerization of amino acids having a pyrrole ring or an imidazole ring are described in WO 97/30975. Polymers obtained by polymerization of various β-amino acids are described in particular by Stigers, Kimberley et al., [“Designed molecules that fold to mimic protein secondary structures”, Current Opinion in Chemical Biology, Vol. 3, No. 6, December 1999 (1999-12)] by Samuel H. Gellman, [“Foldamers A Manifesto”, Acc Chem. Res. (1998) 31(4) 173-180] or by Dieter Seebach et al., [“Beta-peptides A surprise at every turn”, Chem. Commun. (Cambridge) (1997) (21) 2015-2222]. It is known that various groups have properties of constrained mimetics of dipeptides or of tripeptides. Such groups are in particular described by Obrecht, et al. 1999). The compounds containing such groups are described as being of use in orienting peptide chains and mimicking constrained β-turn conformations in peptides.

The aim of the invention is to produce artificial polypeptides and artificial proteins analogous to natural polypeptides and natural proteins, in which the structured peptide components, in particular structured in the form of an α-helix, are replaced with nonpeptide oligomers. Said artificial polypeptides and artificial proteins are more stable than their natural analogs, from which they differ in structure, in particular in size.

A subject of the invention is thus oligomers of amino acids, the recurring units of which are nonpeptide constrained mimetics of dipeptides or tripeptides, a method for the production thereof, the use thereof for developing artificial polypeptides and artificial proteins, and the polypeptides and proteins obtained.

An oligomer of the present invention is represented by one of the general formulae:

R¹—(NR′-A-CO)ₙ—OR² (I) or R¹—(NR′-A-CO)ₙ—NR′²R″² (I′)

in which:

the unit —NR′-A-CO— represents a nonpeptide constrained mimetic of a dipeptide or tripeptide fragment, which induces a β-turn;

n is between 2 and 40;

R¹ represents an acyl group R³—CO— or a group R³—O—CO— in which R³ represents a benzyl group, a tert-butyl group or a 9-fluorenylmethyl group;

R² represents H, an alkyl group (preferably a methyl or an ethyl) or a benzyl group;

R′² and R″² represent, independently of one another, H, an alkyl group (preferably a methyl or an ethyl) or a benzyl group;

R′ represents a hydrogen atom, or else

R′ forms a monocyclic group or a polycyclic group with the N atom and the group A, said polycyclic group being a group which may or may not be condensed.

In a particular embodiment, A represents a heterocyclic group which may or may not be aromatic, and which is a monocyclic group or a polycyclic group which may or may not be condensed.

In another embodiment, the group —NR′-A-CO— represents a heterocyclic group which may or may not be aromatic, and which is a monocyclic group or polycyclic group which may or may not be condensed.

The group —NR′-A-CO— may comprise an asymmetrical center which may have an R configuration or an S configuration.

An oligomer of the invention may consist of recurring units —NR′-A-CO— which are all identical. It may also consist of different units which correspond to the definitions above.

As examples of —NR′-A-CO— groups which have properties of nonpeptide constrained mimetics of dipeptides or tripeptides and which are β-turn inducers, mention may be made of the following groups, in which:

R, and where appropriate, R₄ are chosen, independently of one another, from the groups constituting the side chains of α-amino acids, for example H, CH₃—, (CH₃)₂CH—, CH₃—(CH₂)₃— or C₆H₅—CH₂—;

R₅ and R₆ represent, independently of one another, H, CH₃—, or C₆H₅—CH₂—;

R₇ represents H or a phenyl;

R₈ represents H, CH₃—, C₂H₅— or C₆H₅—CH₂—;

the substituents X and Z are defined specifically for each compound which contains them, and

Me represents a methyl group.

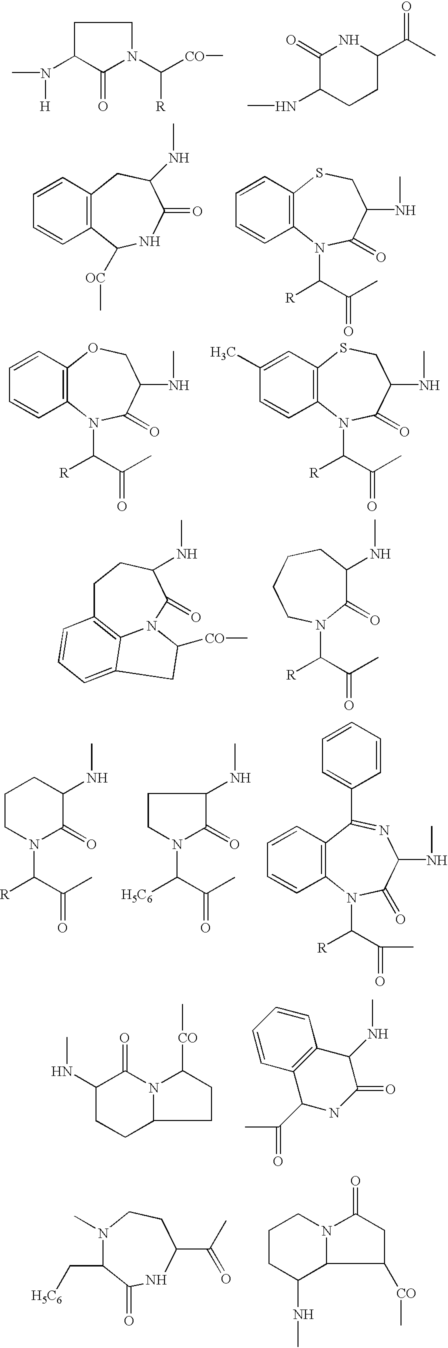

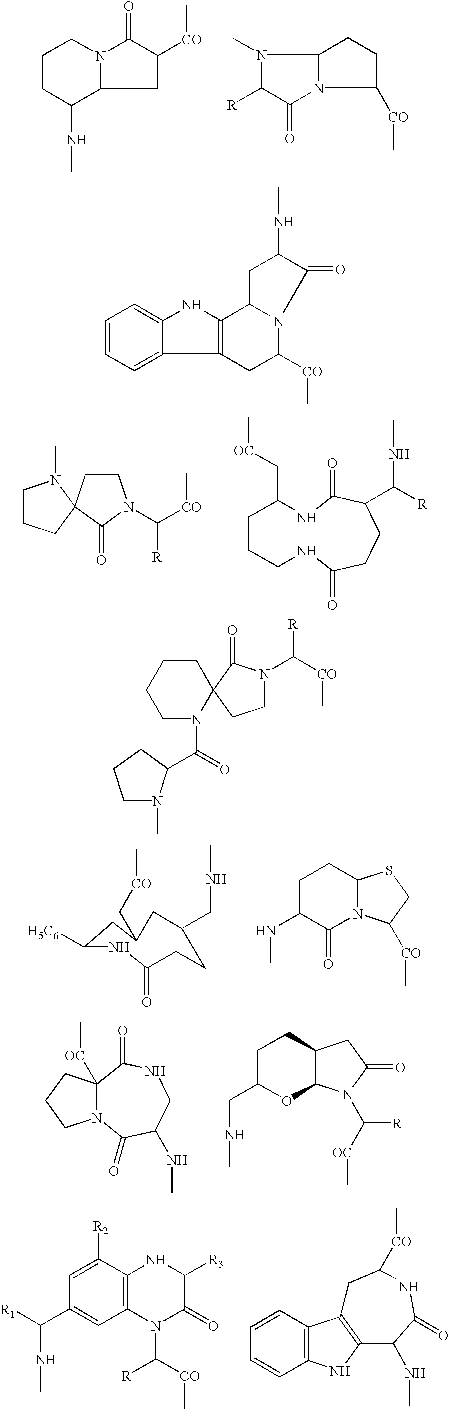

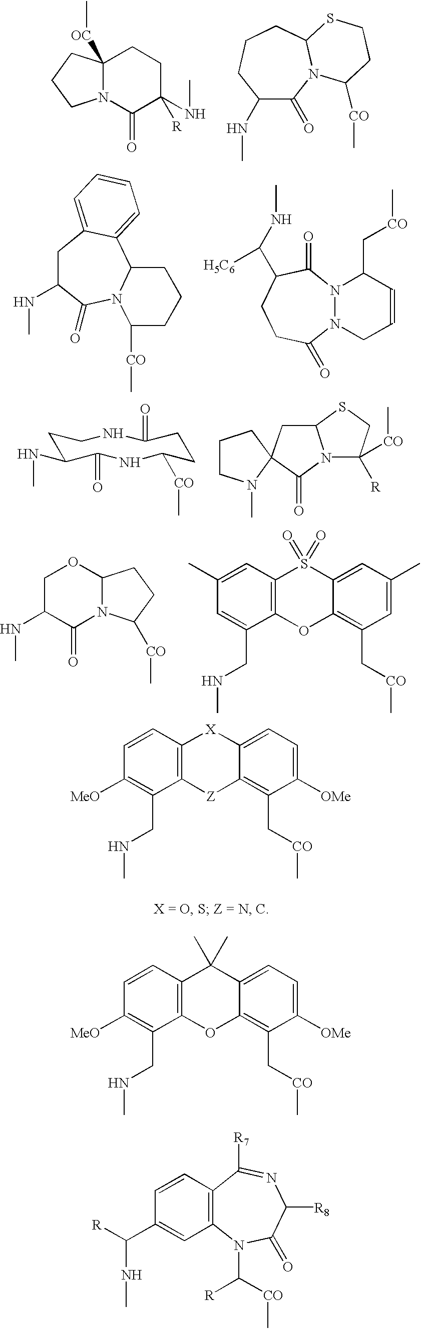

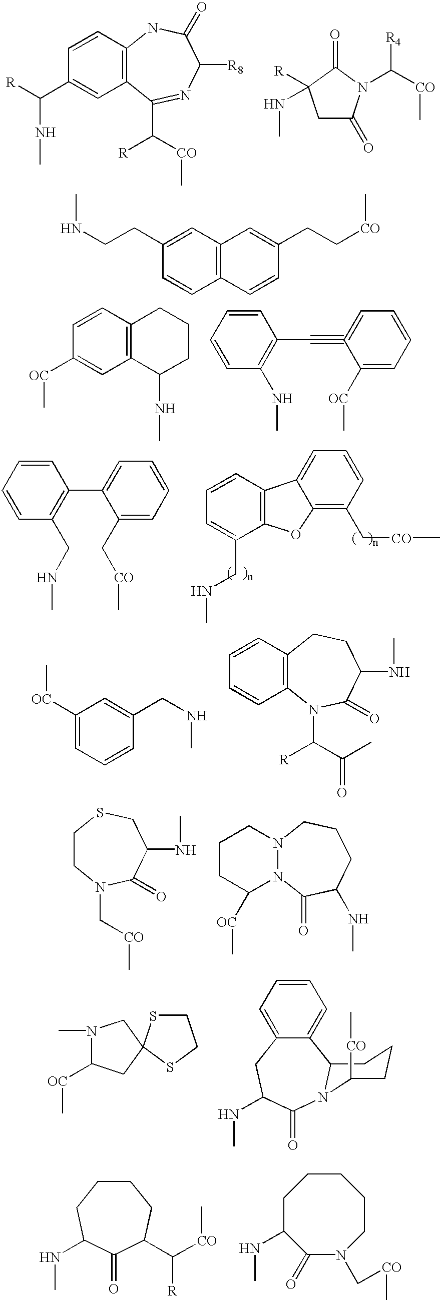

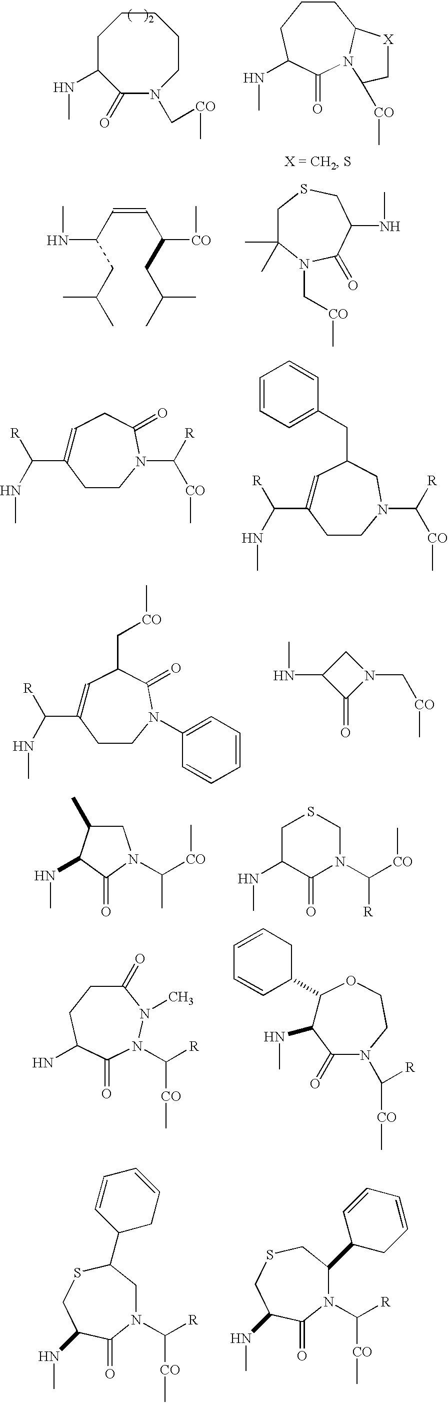

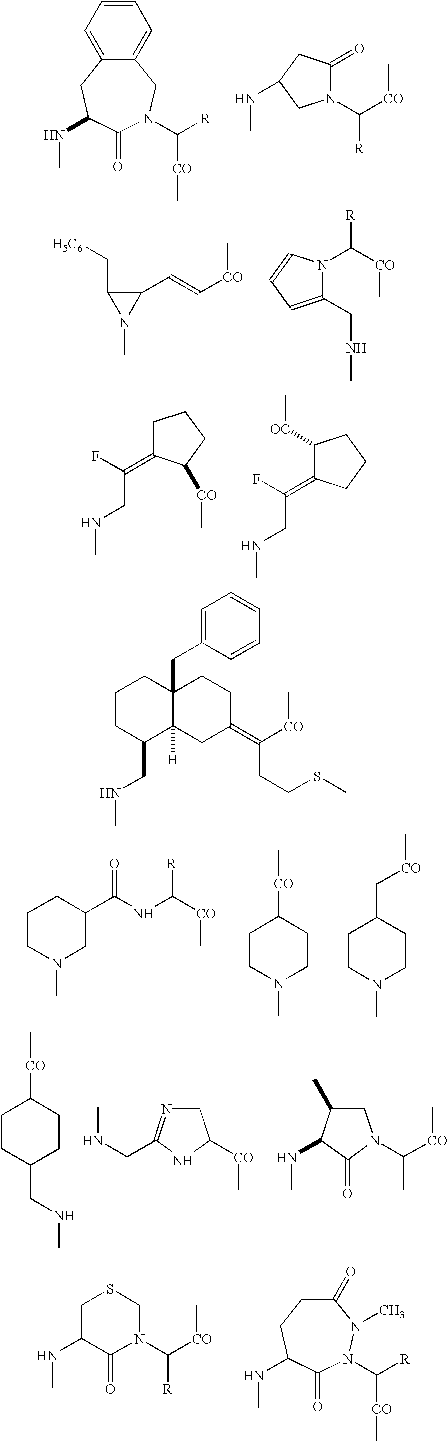

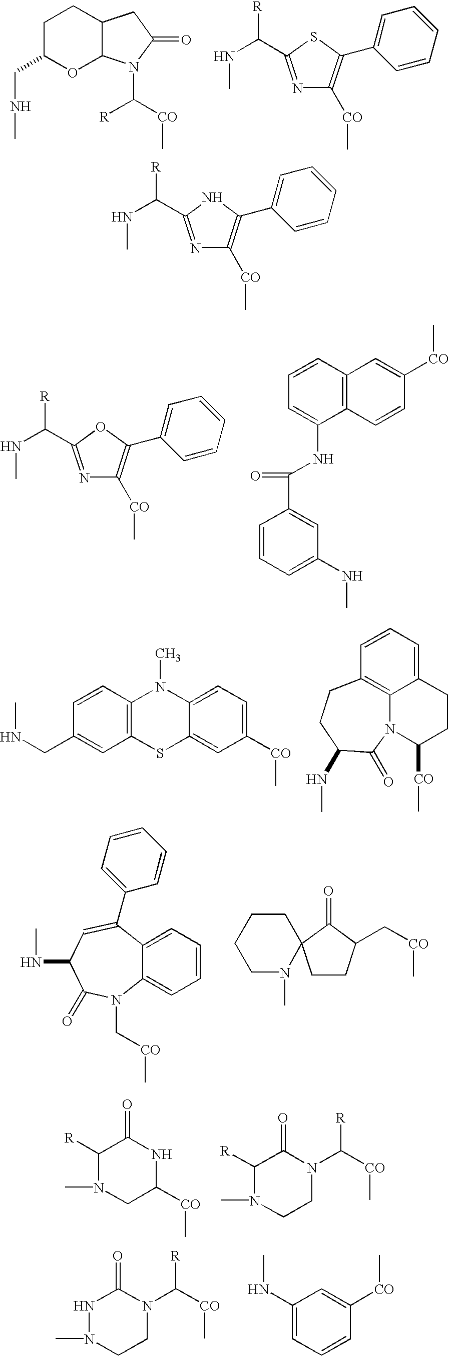

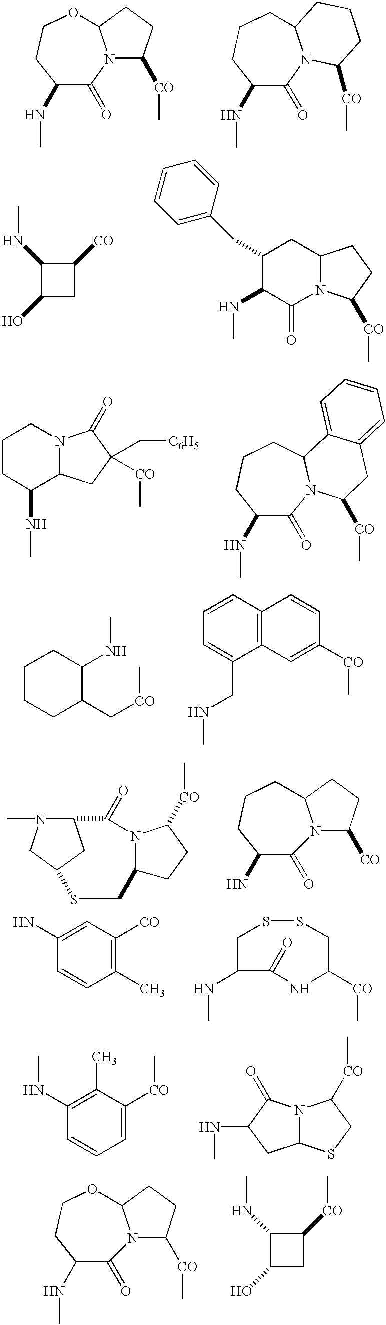

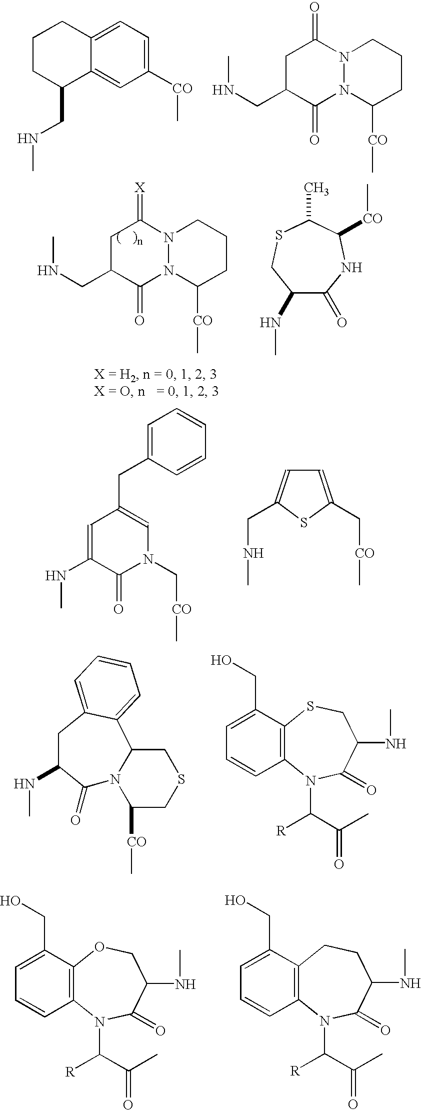

In the compounds above comprising asymmetric centers, said asymmetric centers may have an R or S configuration.

An oligomer (I) or (I′) is obtained by polymerization of at least one amino acid which constitutes a nonpeptide constrained mimetic of a dipeptide or of a tripeptide, which is a β-turn inducer, e and which corresponds to the formula NHR′-A-CO—OH (II) in which the groups R′ and A have the same meaning as in the oligomer (I) or (I′). By way of example of compounds (II), mention may be made of the compounds below:

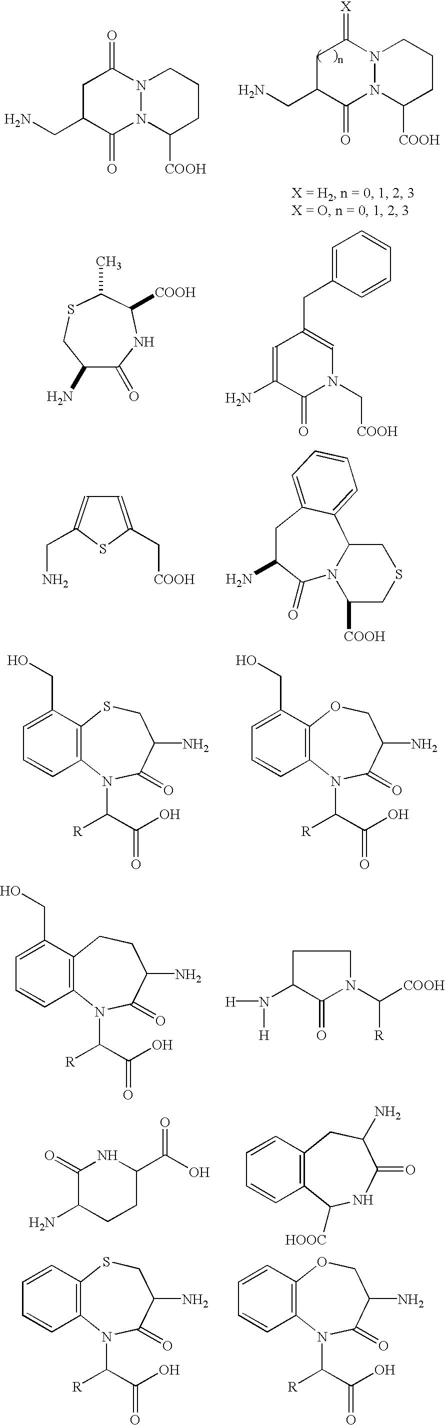

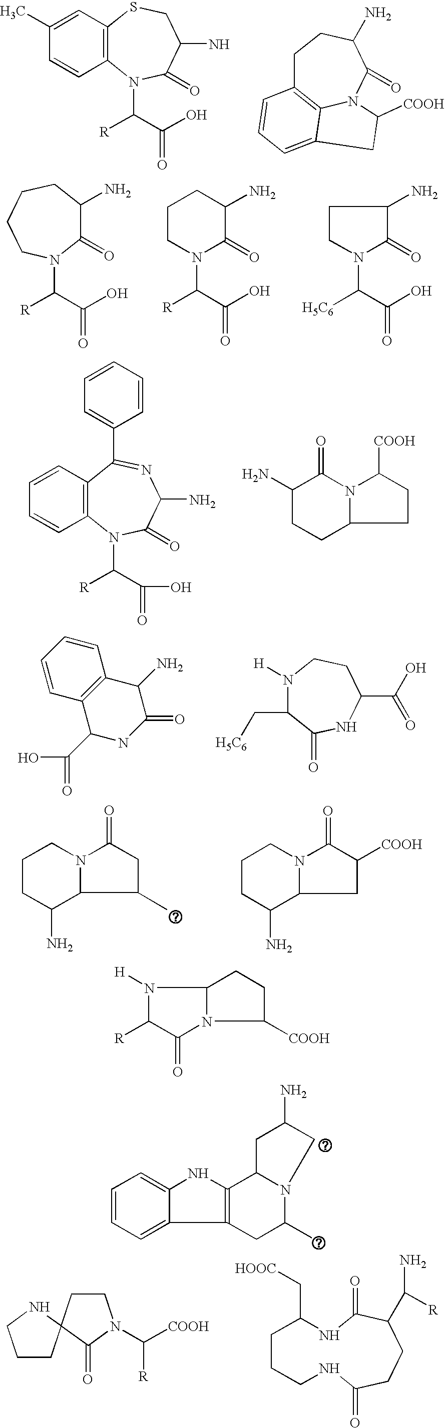

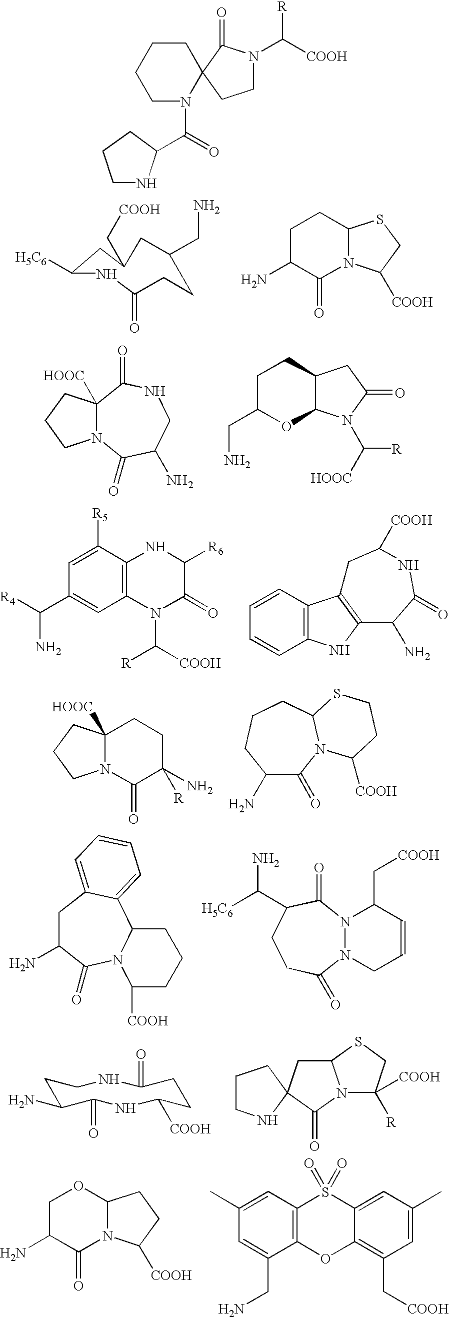

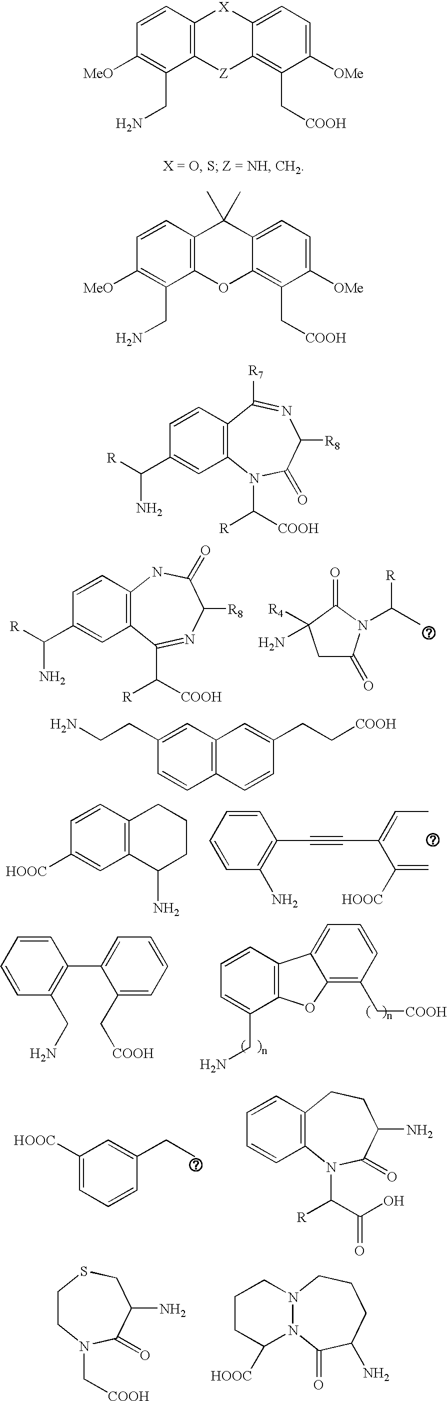

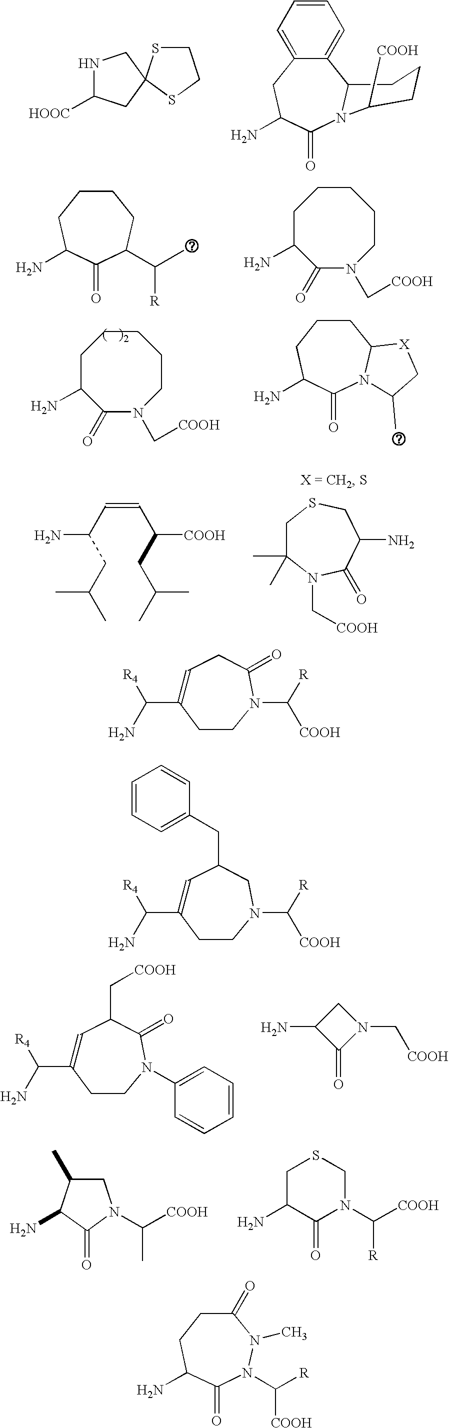

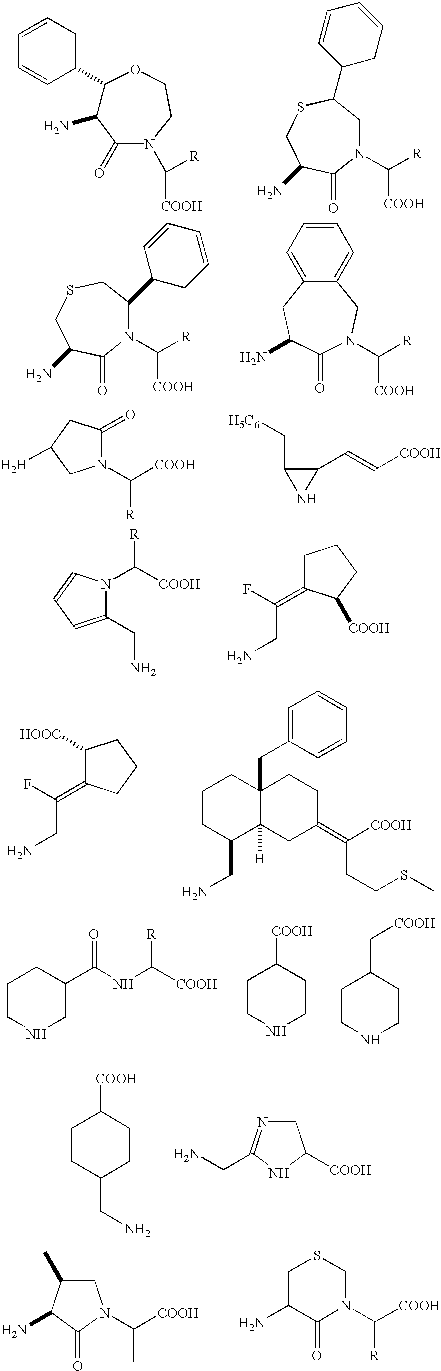

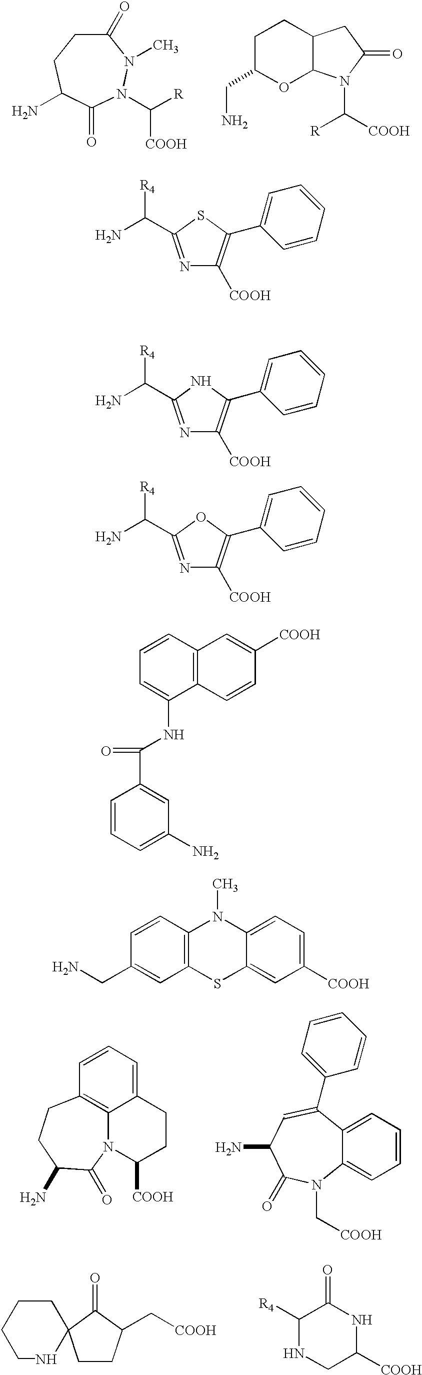

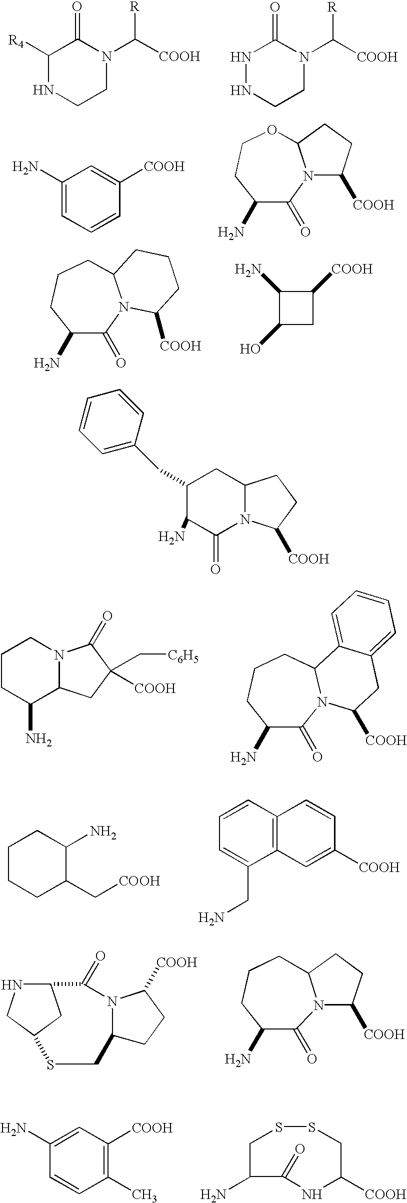

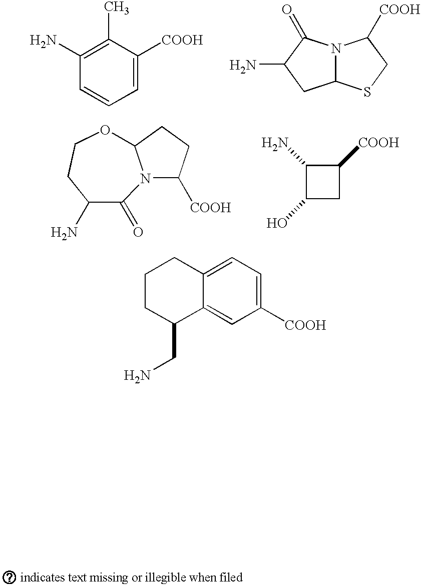

In the compounds above, the substituents R, R₄ to R₈, X and Z have the meaning given above.

In the compounds above comprising asymmetric centers, said asymmetric centers may have an R or S configuration.

These monomers may be synthesized using methods described in the prior art. For example, Amblard et al., J. Med. Chem., 1999, 42, 4193-4201, describe the synthesis of (3S)-[amino]-5-(carbonylmethyl)-2,3-dihydro-1,5-benzothiazepin-4(5H)-one and of other monomers. Other methods are described in Obrecht, mentioned above.

The monomers (II) may be polymerized in solution according to a conventional procedure for peptide syntheses. A solution is formed which contains a monomer (II) protected on its acid function, a monomer (II) which may or may not be identical to the first monomer mentioned and which is protected on its amine function, and a coupling agent, which causes the formation of an amide bond between the two monomers. Then, the N-terminal function of the oligomer obtained is selectively deprotected and another N-protected monomer (II) is condensed with this oligomer so as to obtain a protected oligomer consisting of three monomer units. The synthesis is thus continued with successive steps of selected deprotection of the N-terminal function of the oligomer, followed by coupling with an N-protected monomer, until an oligomer of the desired size is produced. The final oligomer may be ultimately deprotected either selectively on the carboxylic function or on the amine function, or totally.

In an oligomer (I) or (I′), the terminal substituent R₁ may be obtained by using, for the final coupling step, a monomer N-protected with a group R₁, or by carrying out an acylation reaction with the appropriate reagent.

When at least one of the substituents R′₂ or R″₂ is different from H, the terminal group —NR′₂R″₂ of an oligomer (I′) is obtained by choosing, as first N-protected monomer free on its acid function, a compound resulting from reacting a compound (II) with an amide HNR′₂R″₂, corresponding (before protection of the amine function) to the formula NHR′-A-CO—HNR′₂R″₂.

When the substituent R₂ of an oligomer (I) is different from H, the terminal group OR₂ is obtained through the choice of the protective group for the acid function in the first monomer.

An oligomer (I) or (I′) may also be obtained using a method of solid-phase polymerization, according to a conventional strategy of peptide synthesis: a resin carrying amine substituents is functionalized with an amino acid, and the fragment thus attached to the resin is then extended from the C-terminal side to the N-terminal side, the final fragment being protected on its N-terminal end before being separated from the resin. Such a solid-phase method makes it possible to obtain the oligomers (I) and (I′) in which the respective substituents R₂, R′₂ and R″₂ are H.

More precisely, a method of solid-phase polymerization for producing an oligomer of the invention comprises the following steps:

a) a support resin carrying amine substituents is functionalized with a compound H—NR′-A-CO—OH (II′) which corresponds to the definition given for (II) and in which the amino group has been protected beforehand;

b) the fragment thus attached to the support resin is extended from the C-terminal side to the N-terminal side by (n−2) successive reactions for coupling the monomer (II), said monomer (II) being used in excess, various monomers (II) possibly being used in the successive coupling steps;

c) a final reaction is carried out for coupling a monomer protected on its N-terminal function with a group R₁ which is stable under the conditions under which the oligomer must be separated from the support;

d) the oligomer is separated from the support resin.

In the method of solid-phase polymerization, each step for coupling a compound (II) [on the initial support resin in step a) or on the resin carrying a fragment derived from a compound (II) in steps b) and c)] comprises the protection of the amino group of the compound (II), the actual coupling reaction in the presence of a coupling agent, the washing of the product obtained after coupling, and then the deprotection of the amino group of the unit —NR′-A-CO— attached.

The protection of the amino group of the amino acid may be carried out, for example, with a tert-butyloxycarbonyl group (hereafter denoted Boc-) or a 9-fluorenylmethyloxycarbonyl group (hereafter denoted Fmoc) represented by the formula

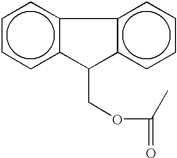

The protection is carried out according to the known methods of the prior art. For example, the protection with the Boc-group may be obtained by reacting the amino acid with di-tert-butylpyrocarbonate (Boc₂O)

For step a), any resin conventionally used in peptide syntheses may be used as support resin. By way of example, mention may be made of a 4-methylbenzydrylamine.HCl resin (hereinafter denoted MBHA.HCl) or a “Merrifield” resin, which is a copolymer of styrene and divinylbenzene functionalized with chlorobenzyl. The two resins are commercial resins distributed, in particular, by the companies Novabiochem and Bachem. Mention may also be made of the PAL-PEG-PS resins, which are 5-(4-aminomethyl-3,5-dimethoxyphenoxy)valeric acid—polyethylene glycol—polystyrene copolymers.

The coupling agent may be chosen from the conventional coupling agents used in peptide synthesis. Mention may be made, for example, of:

| | |
|---|---|
| benzotriazol-1-yloxytris- (dimethylamino)- phosphonium hexaflurophosphate (hereinafter denoted BOP) | 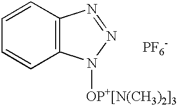 |
| dicyclohexylcarbodiimide (hereinafter denoted DCC) | 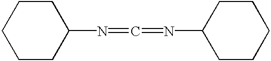 |
| in the presence of 1- hydroxybenzotriazole (hereinafter denoted HOBT) | 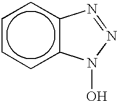 |
| diispropylethylamine (hereinafter denoted DIEA) | 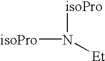 |
| O-(benzotriazol-1-yl)- 1,1,3,3-tetramethyluronium hexafluorophosphate (hereinafter denoted HBTU) | 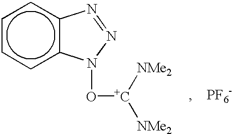 |

For the coupling, use may also be made of the symmetric anhydrides preformed from an N-protected monomer (II) and DCC.

The products obtained after each coupling phase may be washed with the solvents conventionally used in solid-phase peptide syntheses. By way of example, mention may be made of dimethylformamide (DMF), methanol and dichloromethane (DCM).

The reagent used for the deprotection of the amino group after a coupling step depends on the protecting agent used. For example, if the protection is carried out with a Boc group, the deprotection is advantageously carried out using a solution of trifluoroacetic acid (TFA). If the protection is carried out with a Fmoc-group, the deprotection may be carried out with piperidine. In general, the protective groups and the deprotecting reagents used in a known manner in solid-phase peptide syntheses may be used in the synthesis of the oligomers of the present invention.

The oligomer may advantageously be separated from the resin by treatment with an acid. By way of example, mention may be made of trifluoroacetic acid or hydrofluoric acid in the presence of anisole, depending on the type of resin used. Under these conditions, the protection of the amino group before separation from the resin is carried out with a urethane group which is stable under the acid cleavage conditions or with an acyl group which is stable under the same conditions. When the separation is carried out with trifluoroacetic acid, the amino group is protected, for example, with an Fmoc group defined above.

The solid-phase preparation of the oligomers is advantageously carried out in an automatic synthesizer conventionally used for synthesizing peptides on a solid support. In this type of machine, the succession of the various coupling, washing and deprotecting operations is managed by a computer.

The inventors have found that an oligomer (I) or (I′) of the invention, which is obtained by coupling several molecules of the same amino acid monomer (II) or by coupling different monomers (II), forms an organized and rigid structure, for example a helical structure, which can advantageously replace the α-helical structuring fragment(s) of a natural protein or of a natural polypeptide.

A subject of the present invention therefore also consists of an artificial protein or an artificial polypeptide which is analogous to a natural protein or a natural polypeptide, and of a method for the preparation thereof.

The method for preparing an artificial protein or an artificial polypeptide which is analogous to a natural protein or a natural polypeptide consists in carrying out solid-phase peptide synthesis coupling reactions. It is characterized in that, in the succession of reactions for coupling the α-amino acids constituting the natural polypeptide or protein, one or more α-amino acid sequences are replaced with an oligomer (I) or (I′), the length of which is equivalent to that of the α-amino acid sequence replaced. In a particular case, the α-amino acid sequence constituting the α-helix of the natural peptide or protein is replaced with an oligomer (I) or (I′).

The oligomer is prepared beforehand according to the method of solid-phase polymerization as described above, and it is used in a form in which the N-terminal amine function is protected with a protective group which withstands the conditions under which the oligomer will be separated from the support resin. By way of example of such a protective group, mention may be made of the 9-fluorenylmethoxycarbonyl (Fmoc) group.

An artificial polypeptide or protein which is analogous to a given natural polypeptide or protein comprises one or more structuring fragments, for example helical structuring fragments, and one or more peptide fragments. It is characterized in that the peptide fragment(s) is (are) identical to those of the corresponding natural polypeptide or protein, and in that the structuring fragment(s) consist(s) of a fragment of an oligomer (I) or (I′), the length of which is substantially identical to that of the α-helical structuring component of the natural polypeptide or protein.

By way of example, mention may be made of an artificial protein analogous to human CRF or hCRF (“human corticotropin releasing factor”). Such an artificial protein has a structure analogous to that of CRF, in which the central component forming the α-helix, which is a structuring component without biological activity, has been replaced with a sequence —(NR′-A-CO)ₙ—, in which n is 8 or 9 and —NR′-A-CO— is a -DBT- fragment derived from (3S)-[amino]-5-(carbonylmethyl)-2,3-dihydro-1,5-benzothiazepin-4(5H)-one and represented by the formula

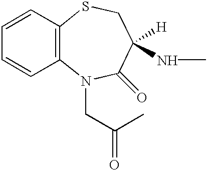

Mention may also be made of another artificial protein analogous to hCRF, in which the central component forming the α-helix has been replaced with a sequence —(NR′-A₁-CO)₂₀— in which —NR′-A₁-CO— is a fragment represented by the formula

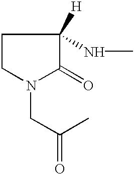

These artificial proteins, analogous to human CRF, exhibit a certain affinity for human CRF receptors.

The present invention is described in greater detail using the following examples, which are given by way of illustration and to which the invention is not limited.

### EXAMPLE 1

### Preparation of a Boc-(DBT)₅-MBHA Resin

The polymerization was carried out in solid phase using an Applied 433 automatic synthesizer, employing a Boc strategy, using:

as amino acid (II): (3S)-[amino]-5-(carbonylmethyl)-2,3-dihydro-1,5-benzothiazepin-4(5H)-one, hereinafter denoted H-DBT-OH, in which the amine function has been protected with a Boc group defined above,

as support resin: 2 g of MBHA.HCl resin substituted at 0.8 mmol/g with amine functions (marketed by Novabiochem).

Using the substituted MBHA resin, the following operation sequence was carried out 5 times:

protection of the amine function of 1.25 eq. of H-DBT-OH by reaction with di-tert-butylpyrocarbonate, Boc₂O, so as to obtain 704 mg (1.25 equivalent) of Boc-DBT-OH;

coupling in dimethylformamide (DMF), using HBTU (758 mg) as coupling agent in the presence of DIEA (0.62 ml), at pH 8-9;

washing of the product obtained, once in DMF, and then twice in methanol and twice in dichloromethane (DCM);

deprotection by treatment with a mixture of trifluoroacetic acid (TFA), dichloromethane (DCM) and ethanedithiol (EDT) in the following proportions: TFA/DCM/EDT (40/60/2), for 2 min, then filtration, and then a second treatment for 28 min with the same TFA/DCM/EDT mixture, the deprotection being omitted after the final coupling step.

An MBHA resin carrying Boc-(DBT)₅-fragments, hereinafter denoted Boc-(DBT)₅-MBHA resin, was thus recovered.

### EXAMPLE 2

### Preparation of an Ac-(DBT)₅-NH₂ Oligomer

250 mg of a Boc-(DBT)₅-MBHA resin obtained according to the procedure described in example 1 were placed in a reactor. The Boc-group was eliminated using a solution consisting of a 40/60/2 TFA/DCM/EDT mixture, in two steps lasting 2 min and 28 min, respectively, separated by a filtration. 5 ml of a 50/50 solution of acetic anhydride Ac₂O in DCM were then added and the mixture was stirred at ambient temperature for 1 hour. The Ac-(DBT)₅-MBHA resin obtained was washed once in DMF, and then twice in methanol and twice in dichloromethane (DCM), and then dried under vacuum. The dried resin was then placed under vacuum.

To obtain the free oligomer, the dried Ac-(DBT)₅-MBHA resin was introduced into a Teflon reactor containing anisole (1 ml/g of resin), followed by introduction of anhydrous hydrofluoric acid, by distillation, into the reactor (10 ml of HF/g of resin). The reaction medium was stirred at 0° C. for 1 hour, and then the HF was eliminated by distillation, anhydrous ether was introduced and the mixture was filtered, this operation sequence being repeated several times.

The crude product was extracted with dimethyl sulfoxide (DMSO), water was added to it and then the mixture was lyophilized, and a flaky white product was obtained. 100 mg of this product were purified by preparative HPLC (high pressure liquid chromatography) and the purified product was analyzed by mass spectrometry [m/z 616.17 (double-charged, M+H⁺)].

The HPLC retention time (Rt) is 15.3 min (UV detection 214 nm, gradient: 0% (A) to 100% (B) in 25 min), which confirms the purity of the product obtained.

### EXAMPLE 3

### Preparation of Ac-(DBT)ₙ-NH₂ Oligomers, n>5

A resin obtained according to the procedure described in example 1 was subjected to two additional protection/coupling/washing/deprotection sequences as described in example 1, and a resin carrying Boc-(DBT)₇-fragments was obtained. 250 mg of this resin were treated according to the procedure of example 2, so as to finally obtain the oligomer Ac-(DBT)₇—NH₂.

In the same way, the oligomer Ac-(DBT)₉-NH₂ and the oligomer Ac-(DBT)₁₁—NH₂ were prepared by subjecting a resin obtained according to the procedure described in example A, respectively to 4 and 6 additional protection/coupling/washing/deprotection sequences, and then treating each of the resins obtained, according to the procedure of example 2.

The various oligomers were analyzed in the same way as the Ac-(DBT)₅-NH₂ oligomer. The results are as follows:

Ac-(DBT)₇-NH₂ m/z 849.9 (double-charged, M+H⁺) HPLC Rt=28.4 min (UV detection 214 nm, gradient: 0% (A) to 100% (B) in 50 min)

Ac-(DBT)₉-NH₂ m/z 1084.5 (double-charged, M+H⁺) HPLC Rt=32.2 min (UV detection 214 nm, gradient: 0% (A) to 100% (B) in 50 min)

Ac-(DBT)₁₁-NH₂ HPLC Rt=33.3 min (UV detection 214 nm, gradient: 0% (A) to 100% (B) in 50 min)

### EXAMPLE 4

### Synthesis of H-(DBT)ₙ-OH Oligomers

Various H-(DBT)ₙ-OH oligomers were prepared for n=2, 3, 5, 7 and 9. A method similar to that of examples 1 and 2 was carried out using:

a Merrifield resin carrying 0.66 mmol of DBT per g of resin,

2.5 equivalents of Boc-DBT-OH per coupling step

BOP as coupling agent.

The Merrifield resin carrying 0.66 mmol of DBT per g of resin was obtained in the following way. 2.5 g (7.1 mmol) of Boc-DBT-OH were dissolved in 20 ml of 95% ethanol, and water was added up to the precipitation limit, followed by 1.17 g (3.55 mmol) of CS₂CO₃. The pH was maintained at 7 and the solution was stirred for 30 min at ambient temperature. After evaporation of the ethanol, the cesium salt (Boc-DBT-O⁻Cs⁺ obtained was lyophilized. 3.32 g (6.80 mmol) of this salt were added to 3 g of a Merrifield resin functionalized with chlorobenzyl, from 1 to 2 mmol/g (marketed by the company Novabiochem), in 20 ml of DMF. The reaction medium was stirred at 60° C. for 48 h, and then the resin obtained was separated by filtration, washed with DMF, DCM, MeOH and water, and again with DCM, and then dried under vacuum. The rate of substitution of the resin with Boc-DBT-O-groups is 0.66 mmol/g of resin.

For an oligomer containing n DBT units, the coupling reaction on the Merrifield resin functionalized with DBT was carried out (n−1) times. At the end of the polymerization, the Boc groups were eliminated and the resin was cleaved in the same way as in example 2. The oligomers were purified by HPLC on a C18 column, and their purity was controlled by HPLC on a Waters 150×4.6 mm, 100 Å, 5 μm C18 column. The characterization was carried out by mass spectrometry. The results are as follows:

H-(DBT)₅-NH₂ HPLC Rt=18.2 min (UV detection 214 nm, gradient: 30% (A) to 60% (B) in 20 min)

H-(DBT)₃-NH₂ HPLC Rt=11.6 min (UV detection 214 nm, gradient: 30% (A) to 60% (B) in 15 min)

H-(DBT)₂-NH₂ HPLC Rt=5.4 min (UV detection 214 nm, gradient: 30% (A) to 40% (B) in 10 min)

The products obtained were also characterized by proton ¹H NMR at 300 MHz.

### EXAMPLE 5

### Synthesis of an Artificial Protein Analogous to hCRF H-Ser-Glu-Glu-Pro-Pro-(DBT)₈-Lys-Leu-Met-Glu-Ile-Ile-NH₂

hCRF ((human) corticotropin releasing factor) is a 41 amino acid amidated peptide. The α-helical component of hCRF (consisting of 30 amino acids) was replaced with a -(DBT)₈-oligomer fragment, so as to obtain the artificial protein of the present example.

During a first step, the oligomer Fmoc-(DBT)₈-OH was prepared using a method of solid-phase polymerization on a Merrifield resin functionalized with 0.66 mmol of DBT per g of resin obtained according to the method described in example 4. 0.76 g of this functionalized resin was placed in the reactor of an automatic synthesizer and 6 successive couplings were carried out under conditions similar to those described in example 1, using, in each coupling, 4 equivalents (0.528 g, 2 mmol) of Boc-DBT-OH and 20 ml of a 1M HOBT and DCC solution in DMF as coupling agent. After each coupling step, the amine function of the fragment attached to the resin was deprotected with a solution of TFA. A seventh coupling reaction was carried out under the same conditions using Fmoc-DBT-OH instead of Boc-DBT-OH. Next, the resin obtained was dried under vacuum and then treated with HF in the presence of anisole according to the procedure described in example 2. After elimination of HF by distillation, the residue was washed several times with ether, and filtered in the presence of the resin. The crude product recovered was dissolved in DMSO and precipitated with water, washed with ether and dried under vacuum over P₂O₅, which made it possible to obtain 400 mg of crude product. 100 mg of this crude product were purified by preparative HPLC and the purified Fmoc-(DBT)₈-OH product obtained was analyzed by mass spectrometry. HPLC Rt=34.0 min (UV detection 214 nm, gradient: 0% (A) to 100% (B) in 50 min).

The artificial protein was then synthesized in an automatic synthesizer, employing a Boc strategy, using an MBHA resin which makes it possible to generate the C-terminal amide function, according to the following synthesis scheme, in which the DBT fragment represents the fragment of the amino acid involved in the coupling reaction. The amino acids successively coupled are given in the table below.

|Cycles of:
|coupling: Boc-DBT-OH, 4 eq.
|DCC/HOBt, 60 min
|deprotection: TFA, 30 min
↓
H-Lys(Z)-Leu-Met-Glu(Ochx)-Ile-Ile-NH-resin
|
|Double coupling: Fmoc-(DBT)₈-OH,
|BOP/DTEA, 12 hours
|deprotection: piperidine/DMF (20/80)
↓
H-(DBT)₈-Lys(Z)-Leu-Met-Glu(Ochx)-Ile-Ile-NH-resin
|Cycles of:
|coupling: Boc-DBT-OH, 4 eq.
|DCC/HOBt, 60 min
|deprotection TPA, 30 mm
↓
H-Ser (Bzl)-Glu (Ochx)-Glu (Ochx)-Pro-Pro-((DBT)₈-Lys(Z)-Leu-Met-Glu(Ochx)-Ile-Ile-NH-resin
|
|HF/anisole (10/1)/° C.
|60 min
↓
H-Ser-Glu-Glu-Pro-Pro-(DBT)₈-Lys-Leu-Met-Glu-Ile-Ile-NH₂

0.625 g of MBHA.HCl resin substituted at 0.8 mmol was placed in the column of the reactor of an automatic synthesizer functioning with a Boc strategy, as were 4 equivalents of each of the required amino acids, corresponding to the following amounts:

| Amino acid | Amount (mg) |
|---|---|
| Boc-Ile-OH | 463 |
| Boc-Ile-OH | 463 |
| Boc-Glu (Ochx)-OH | 659 |
| Boc-Met-OH | 498 |
| Boc-Leu-OH.H₂O | 499 |
| Boc-Lys (Z)-OH | 761 |
| Fmoc- (DBT)₈-OH | 1056 |
| Boc-Pro-OH | 430 |
| Boc-Pro-OH | 430 |
| Boc-Glu (Ochx)-OH | 659 |
| Boc-Glu (Ochx)-OH | 659 |
| Boc-Ser (Bzl)-OH | 591 |

In the table above, “chx” means “cyclohexyl”, “Z” means “benzyloxycarbonyl” and “Bzl” means “benzyl”.

During a first step, the peptide-resin H-Lys(Z)-Leu-Met-Glu(Ochx)-Ile-Ile-NH-resin was synthesized automatically by carrying out 6 successive coupling steps lasting 60 min, using DCC in HOBt as coupling agent, each amino acid being protected with a Boc group, each coupling reaction being followed by a step of deprotection with TFA for 30 min.

During a second step, the coupling of the Fmoc-(DBT)₈-OH was carried out according to the following process. 1056 mg (1 mmol) of H-(DBT)₈-OH oligomer were coupled to the peptide resin H-Lys(Z)-Leu-Met-Glu(Ochx)-Ile-Ile-MBHA resin using a BOP/DIEA mixture as coupling agent, for 12 hours. The reaction product was then treated with a 20% solution of piperidine in DMF so as to eliminate the Fmoc protective group.

During a third step, the extension with the N-protected amino acids was continued in automatic mode so as to obtain the compound Boc-Ser(Bzl)-Glu(Ochx)-Glu(Ochx)-Pro-Pro-(DBT)₈-Lys (Z)-Leu-Met-Glu(Ochx)-Ile-Ile-MBHA-resin.

After the end of synthesis, the compound carried by the resin was dried under vacuum, and was treated with a TFA/DCM/EDT (50/50/2) solution for 2 min and then for 28 min. The resin carrying the compound was then treated with HF in the presence of anisole according to the procedure described above. The compound obtained was purified by HPLC and was analyzed by mass spectrometry as indicated in example 2. The results obtained are as follows:

HPLC Rt=25.0 min (UV detection 214 nm, gradient: 0% (A) to 100% (B) in 50 min).

### EXAMPLE 6

### Synthesis of an Artificial Protein Analogous to hCRF H-Ser-Glu-Glu-Pro-Pro-(DBT)₉-Leu-Met-Glu-Ile-Ile-NH₂

In the present example, the intention was to replace the α-helical component with a -(DBT)₉-oligomer fragment.

The artificial protein was synthesized using a method similar to that of example 5, but preparing an Fmoc-(DBT)₉-OH oligomer beforehand and using the amino acids of the table of example 5, with the exception of Boc-Lys(Z)-OH.

### EXAMPLE 7

### Synthesis of an Fmoc-(A₁)₅-OH Oligomer

During a first step, a Merrifield resin was functionalized with Boc-A₁-OH, so as to obtain Boc-A₁-O-Merrifield. The functionalization was carried out by esterification of a chloromethylated Merrifield resin substituted with from 1 to 2 mmol/g.

The group A₁ corresponds to the formula below:

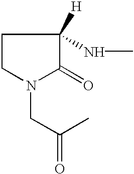

The amino acid (II) used is 3-(S)-amino-1-carbonylmethylpyrrolidin-2-one. The Merrifield resin is a resin marketed by the company Novabiochem.

The fragment was then extended, step by step, from the C-terminal end to the N-terminal end by couplings with BOP and DIEA and successive deprotections. The 15 final mimetic is introduced in the form of Fmoc-A₁-OH, and then the total polymer is cleaved from the support in the form of Fmoc-(A₁)₅-OH, using HF at 0° C. in the presence of anisole. These cleavage conditions make it possible to conserve the N-terminal Fmoc protection.

The synthesis was carried out according to the following scheme:

| | |
|---|---|
| Couplings 1, 2, 3 | Boc-A₁-OH (2 eq.), 1.48 g |
| | BOP, 2.55 g |
| | DIEA 1.98 ml |
| | DCM |
| Coupling 4 | Fmoc-A₁-OH (2 eq.), 2.19 g |
| | BOP, 2.55 g |
| | DIEA 1.98 ml |
| | DMF |
| Washing | 1 × DCM |
| | 2 × MeOH |
| | 2 × DCM |
| Deprotection | TFA/DCM (50/50) 2 min |
| | drying |
| | TFA/DCM (50/50) 28 min |

To carry out the cleavage, the resin (1.1 g) was placed in a Teflon reactor containing 1.1 ml of anisole. After distillation of HF (11 ml) into the reactor, the mixture was stirred at 0° C. for 1 h. The HF was eliminated by distillation and the expected peptide was precipitated with ether and then filtered in the presence of the deprotected resin. The crude product was eluted with a CH₃CN/H₂O/TFA (50/50/0.1) mixture and then lyophilized to produce a white flaky compound. The operation was repeated 3 times, so as to produce 1.84 g of product, which was analyzed by mass spectrometry (ESI), m/z 941.

### EXAMPLE 8

### Preparation of Artificial Protein H-Ser-Glu-Glu-Pro-Pro-(A₁)₂₀-Arg-Lys-Leu-Met-Glu-Ile-Ile-NH₂

The synthesis of the artificial protein H-Ser-Glu-Glu-Pro-Pro-(A₁)₂₀-Arg-Lys-Leu-Met-Glu-Ile-Ile-NH₂ analogous to hCRF was carried out using a PerSeptive automatic synthesizer, on a PAL-PEG-PS resin obtained from the company PerSeptive Biosystems. The mimetics were introduced by blocks of 5 (4×Fmoc-(A₁)₅-OH). The couplings were carried out in the presence of HBTU and DIEA.

The various amino acids used are marketed by the companies Bachem, Propetide and Novabiochem.

0.6 g of Fmoc-PAL-PEG-PS resin substituted at 0.19 mmol/g were placed in the reactor of the synthesizer. 5 equivalents of each amino acid were used for the coupling (double coupling carried out), corresponding to the amounts given in the following table:

| Amino acid | Amount (mg) |
|---|---|
| Fmoc-Ile-OH | 201 |
| Fmoc-Ile-OH | 201 |
| Fmoc-Glu(OtBu)-OH | 248 |
| Fmoc-Met-OH | 212 |
| Fmoc-Leu-OH.H₂O | 201 |
| Emoc-Lys (Boc)-OH | 267 |
| Fmoc-Arg (Pbf)-OH | 370 |
| Fmoc-(A₁)₅-OH × 4 | 214 × 4 |
| Fmoc-Pro-OH | 154 |
| Fmoc-Pro-OH | 154 |
| Fmoc-Glu(OtBu)-OH | 194 |
| Fmoc-Glu(OtBu)-OH | 194 |
| Fmoc-Ser(tBu)-OH | 175 |

In the table above, “OtBu” means tert-butyl ester; “Pbf” means 2,2,4,6,7-pentamethyldihydrobenzofuran-5-sulfonyl; “tBu” means “tert-butyl”.

HBTU and DIEA were used in the form of 0.5 M solutions in dimethylformamide. The Fmoc group was removed by the action of a solution of piperidine in DMF (20/80). 2 equivalents of Fmoc-(A₁)₅-OH (214 mg) were introduced using a manual reactor in the presence of BOP (100 mg) and DIEA (40 μl) in DMF. The Fmoc group was removed with 5 ml of a solution of piperidine in DMF. The deprotection was carried out in 2 steps: 3 min and then 7 min. The resin was washed once with DMF, twice with MeOH and twice with DCM. The final fragment was not deprotected and the resin was replaced in the automatic synthesizer. The Fmoc group of the final amino acid (before cleavage and final deprotection) was removed.

At the end of synthesis, the resin was dried under vacuum and then treated with a TFA/thioanisole solution for two hours.

The scheme for all the reactions is summarized below:

| | Fmoc-PAL-PEG-PS resin |
|---|---|
| | \|Deprotection then |
| | \|Cycles of: |
| PerSeptive | \|coupling: Fmoc-A₁-OH, 5 eq. |
| synthesizer | \|HBTU/DIEA |
| | ↓ |
| H-Arg(Pbf)-Lys(Boc)-Leu-Met-Glu(OtBu)-Ile-Ile-NH-resin | |
| | \|1) deprotection: piperidine/DMF |
| | \|2) 4×double coupling: Fmoc-(A₁)₅-OH, |
| | \|BOP/DIEA |
| | \|3) piperidine/DMF (20/80) |
| | ↓ |
| Fmoc-(A₁)₂₀-Arg(Pbf)-Lys(Boc)-Leu-Met-Glu(OtBu)-Ile-Ile-NH-resin | |
| | \|Cycles of: |
| PerSeptive | \|coupling: Boc-A₁-OH, 5 eq. |
| synthesizer | \|HBTU/DIEA |
| | \|deprotection: piperidine |
| | ↓ |
| H-Ser(tBu)-Glu(OtBu)-Glu(OtBu)-Pro-Pro-(A₁)₂₀-Arg(Pbf)-Lys(Boc)-Leu-Met-Glu(OtBu)-Ile-Ile-NH-resin | |
| | \|TFA/DCM/EDT, 30 min |
| | \|HF/anisole (10/1) 0° C. |
| | ↓60 min |

H-Ser-Glu-Glu-Pro-Pro-(A₁)₂₀-Arg-Lys-Leu-Met-Glu-Ile-Ile-NH₂

1. An oligomer, represented by one of the general formulae:
R₁—(NR′-A-CO)ₙ—OR₂ (I) or R₁—(NR′-A-CO)ₙ—NR′₂R″₂ (I′)
in which:
the unit —NR′-A-CO— represents a β-turn inducing nonpeptide constrained mimetic of a dipeptide or tripeptide fragment;
R₁ represents an acyl group R₃—CO— or a group R₃—O—CO— in which R₃ represents a benzyl group, a tert-butyl group or a 9-fluorenylmethyl group;
R₂ represents H, an alkyl group or a benzyl group;
R′₂ and R″₂ represent, independently of one another, H, an alkyl group or a benzyl group;
R′ represents a hydrogen atom or else R′ forms a monocyclic group or an optionally condensed polycyclic group with the N atom and the group A;
n is between 2 and 40.

2. The oligomer as claimed in claim 1, characterized in that A represents a heterocyclic group which may or may not be aromatic, and which is a monocyclic group or an optionally condensed polycyclic group.

3. The oligomer as claimed in claim 1, characterized in that the group —NR′-A-CO— represents a heterocyclic group which may or may not be aromatic, and which is a monocyclic group or an optionally condensed polycyclic group.

4. The oligomer as claimed in claim 1, characterized in that the group —NR′-A-CO— comprises an asymmetrical center which may have an R configuration or an S configuration.

5. The oligomer as claimed in claim 1, characterized in that the recurring units —NR′-A-CO— are all identical.

6. The oligomer as claimed in claim 1, characterized in that the recurring units —NR′-A-CO— are different.

7. The oligomer as claimed in claim 1, characterized in that the recurring units are chosen from the following groups: 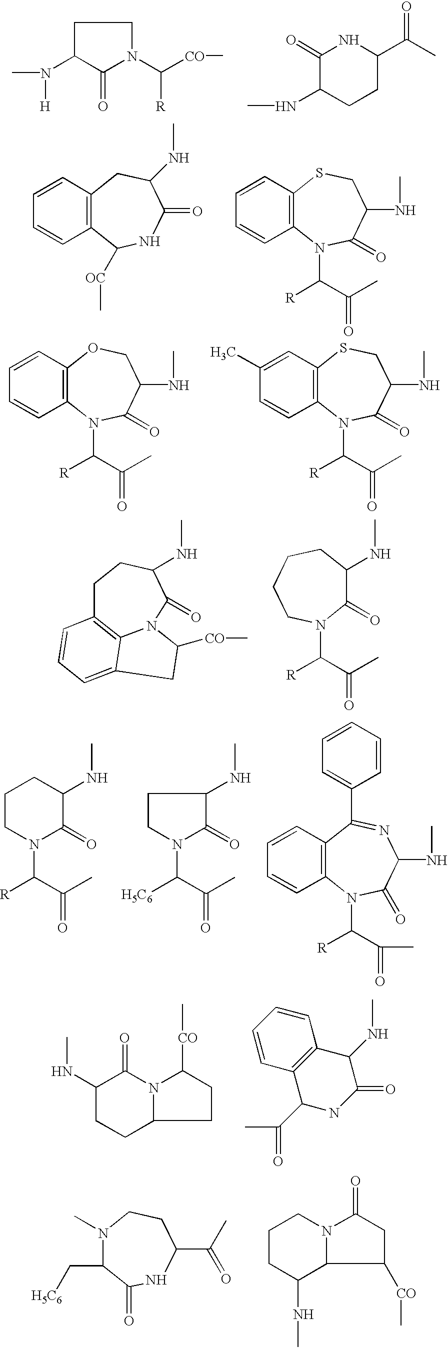 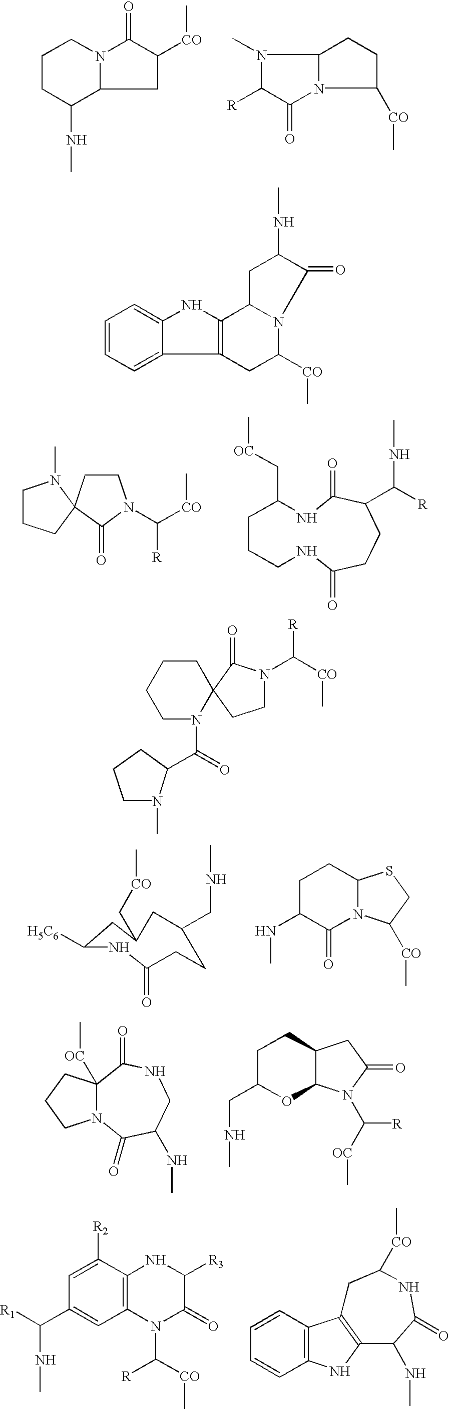 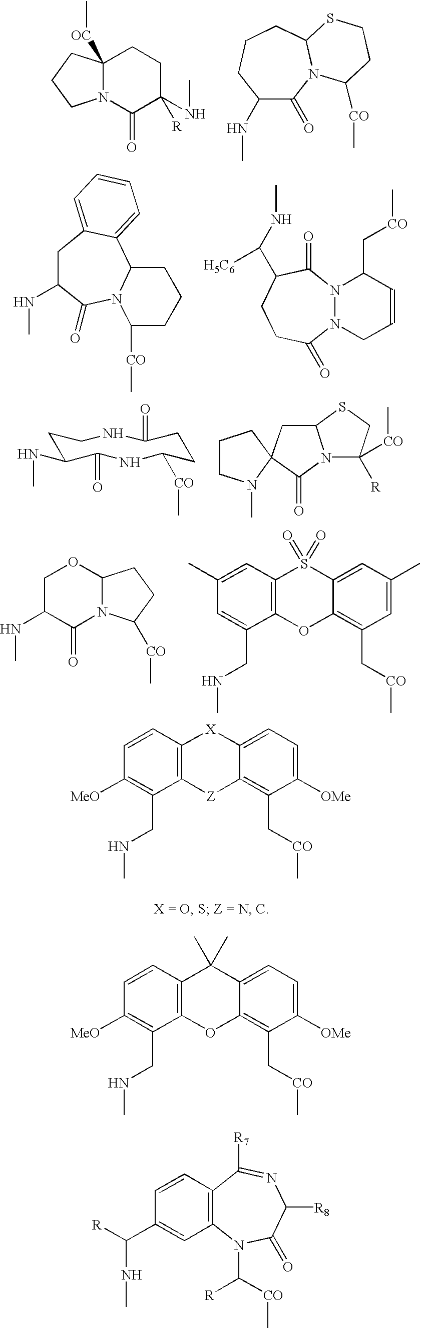 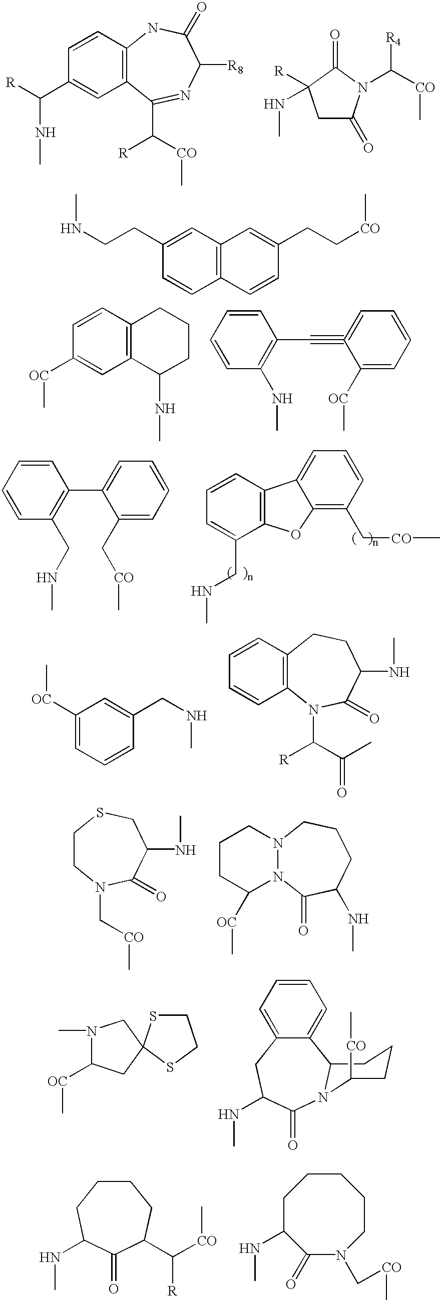 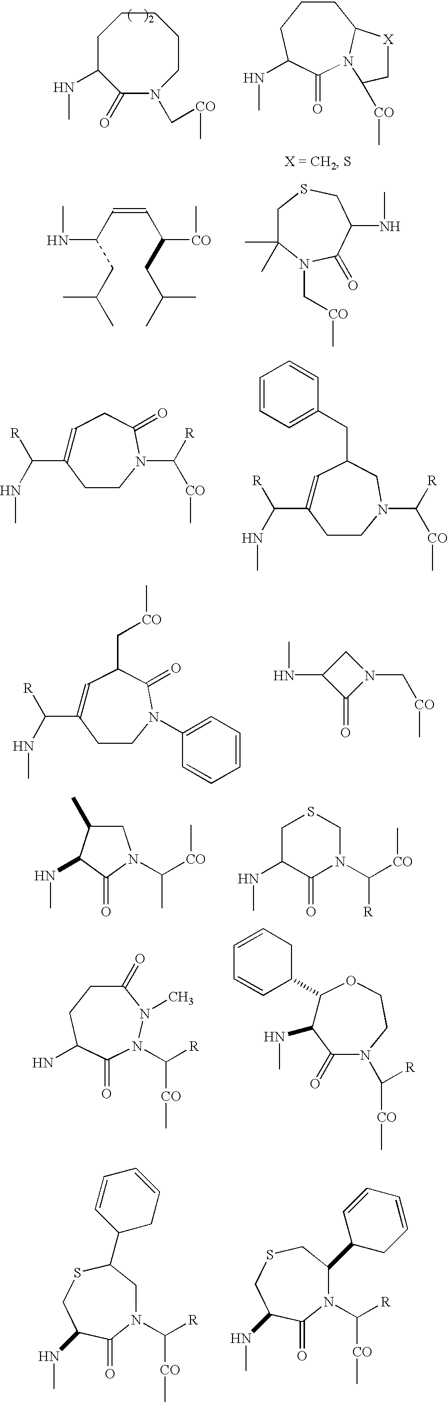 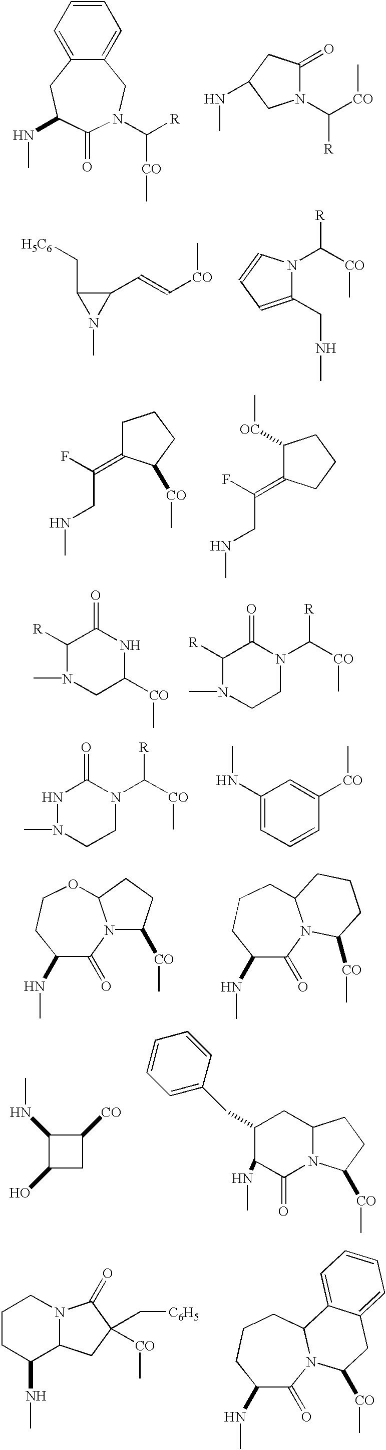 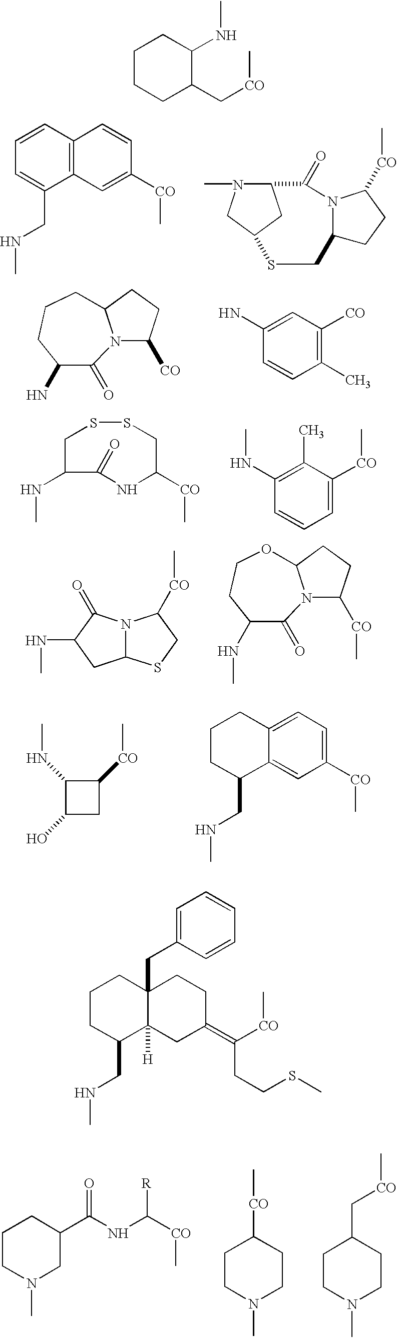 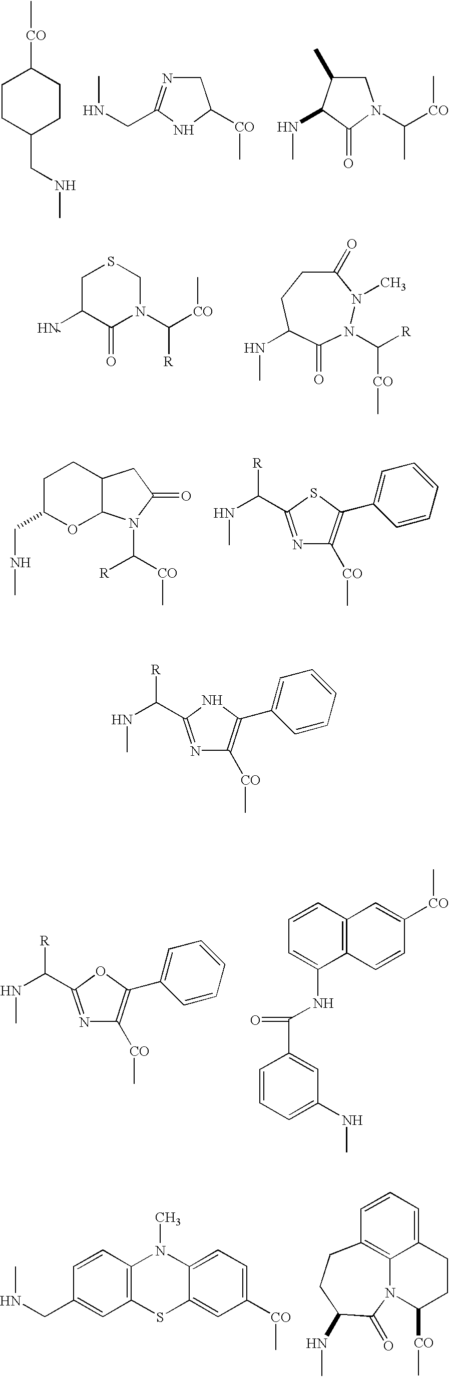 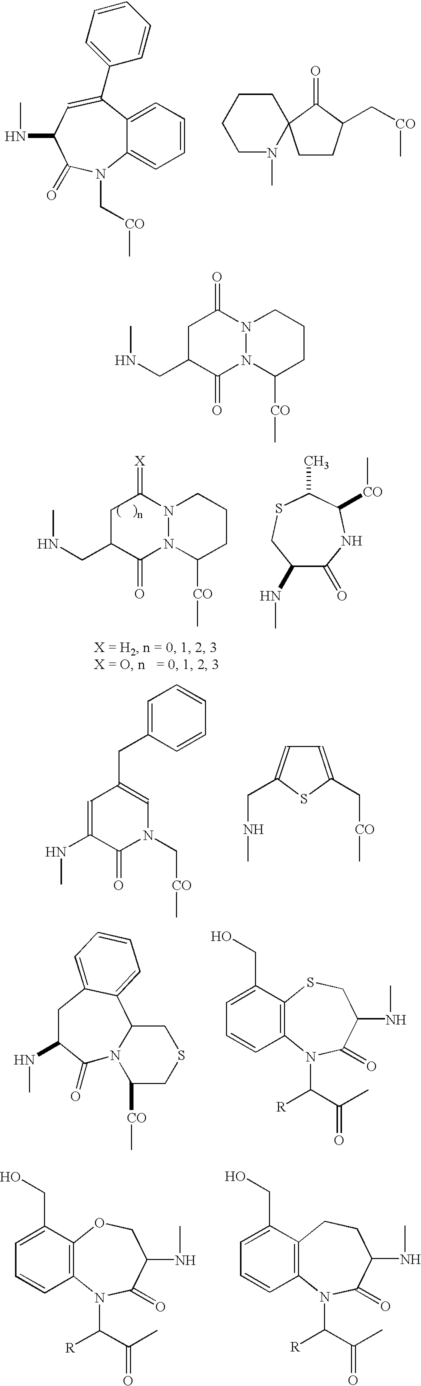
in which:
R, and where appropriate R₄, are chosen, independently of one another, from the groups constituting the side chains of α-amino acids;
R₅ and R₆ represent, independently of one another, H, CH₃— or C₆H₅—CH₂—;
R₇ represents H or a phenyl;
R₈ represents H, CH₃—, C₂H₅— or C₆H₅—CH₂—;
the substituents X and Z are defined specifically for each compound which contains them, and
Me represents a methyl group.

8. The oligomer as claimed in claim 7, characterized in that R, and where appropriate R₄ are chosen, independently of one another, from H, CH₃—, (CH₃)₂CH—, CH₃—(CH₂)₃— or C₆H₅—CH₂—.

9. A method for preparing an oligomer as claimed in claim 1, characterized in that it consists in carrying out a polymerization of at least one amino acid constituting a β-turn inducing nonpeptide constrained mimetic of a dipeptide or of a tripeptide and corresponding to the formula NHR′-A-CO—OH (II), in which R′ represents a hydrogen atom or else R′ forms a monocyclic group or an optionally condensed polycyclic group with the N atom and the group A.

10. The method as claimed in claim 9, characterized in that A represents a heterocyclic group which may or may not be aromatic, and which is a monocyclic group or an optionally condensed polycyclic group.

11. The method as claimed in claim 9, characterized in that the group —NR′-A-CO— represents a heterocyclic group which may or not be aromatic, and which is a monocyclic group or an optionally condensed polycyclic group.

12. The method as claimed in claim 9, characterized in that the group —NR′-A-CO— comprises an asymmetrical center.

13. The method as claimed in claim 9, characterized in that the polymerization is carried out in solution.

14. The method as claimed in claim 9, characterized in that the polymerization is carried out in solid phase, according to a peptide synthesis strategy.

15. The method as claimed in claim 9, characterized in that the compound (II) is chosen from the following compounds, in which:
R, and where appropriate R₄, are chosen, independently of one another, from the groups constituting the side chains of α-amino acids;
R₅ and R₆ represent, independently of one another, H, CH₃— or C₆H₅—CH₂—;
R₇ represents H or a phenyl;
R₈ represents H, CH₃—, C₂H₅— or C₆H₅—CH₂—;
the substituents X and Z are defined specifically for each compound which contains them, and
Me represents a methyl group;

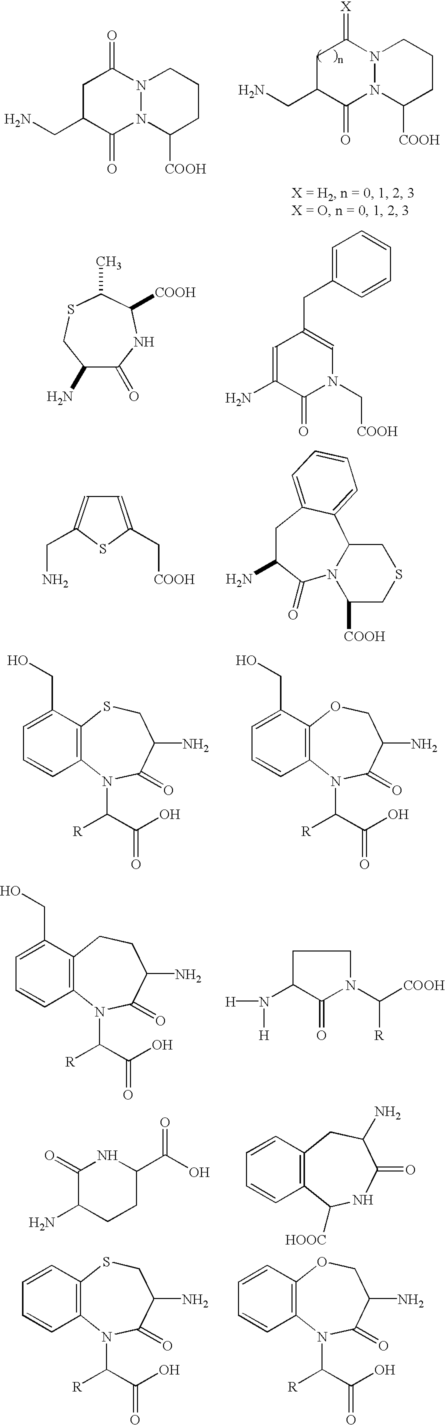

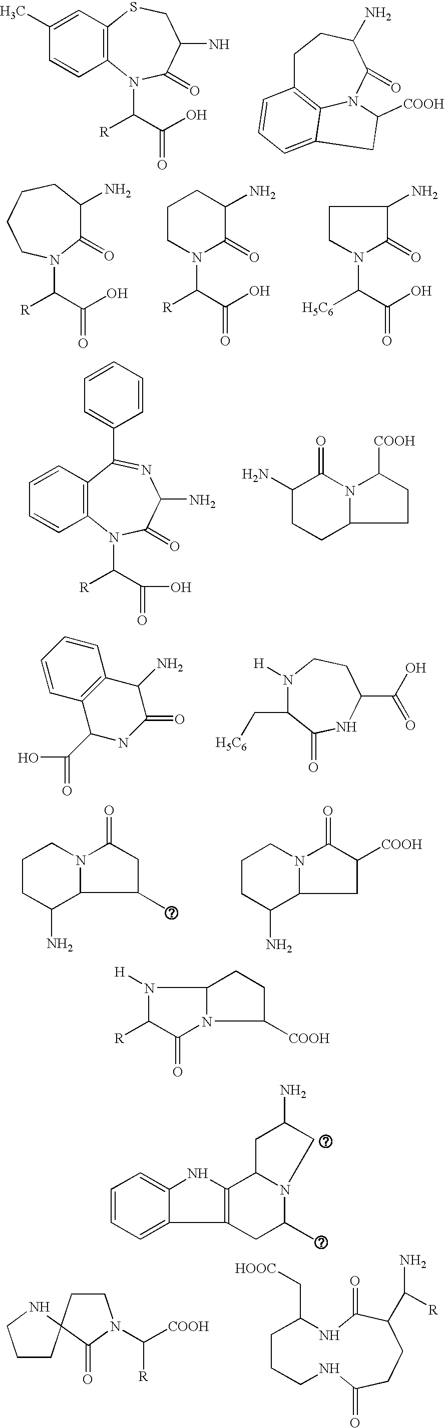

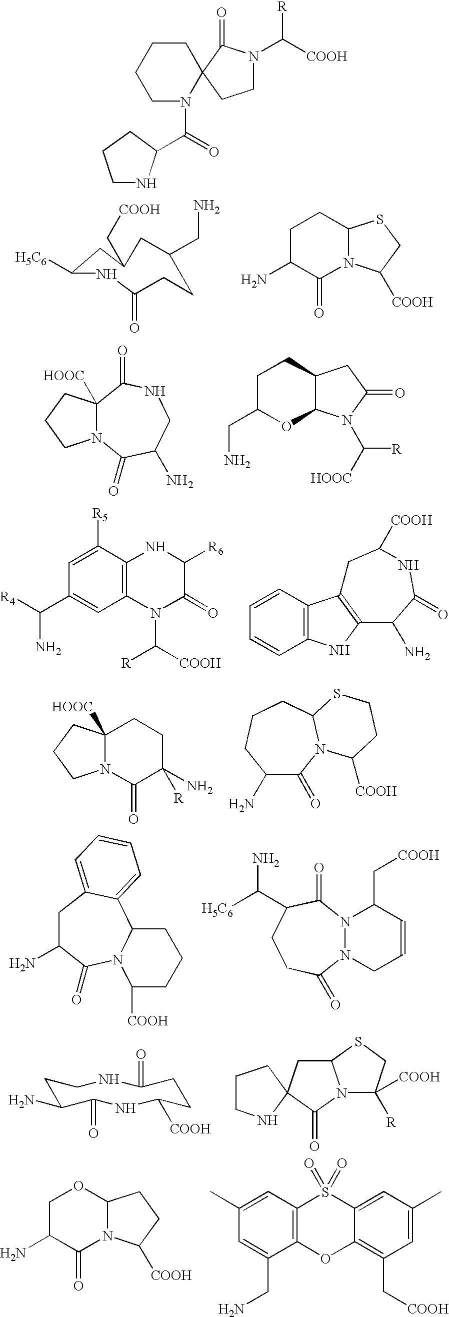

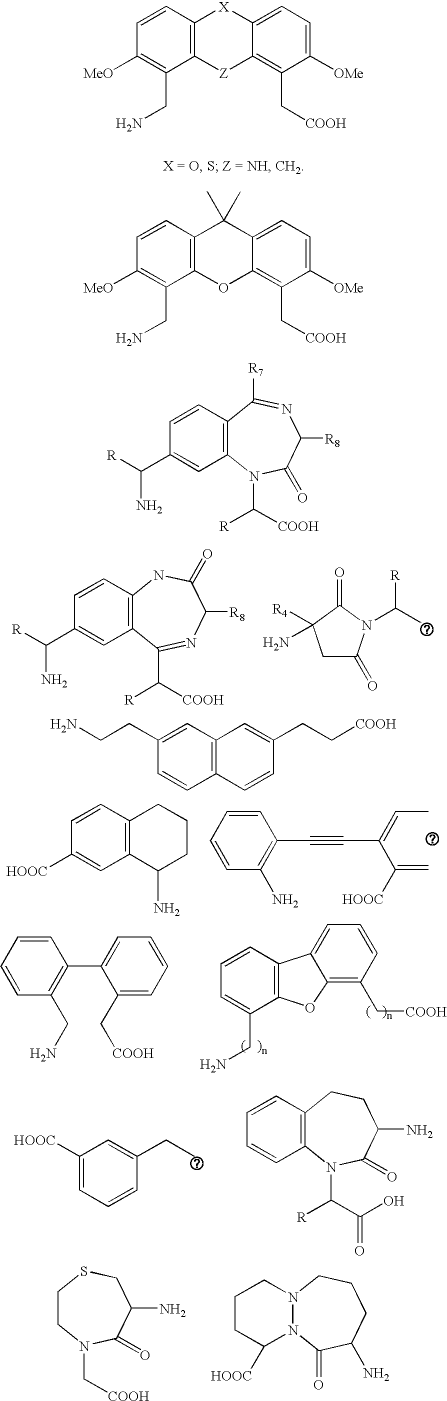

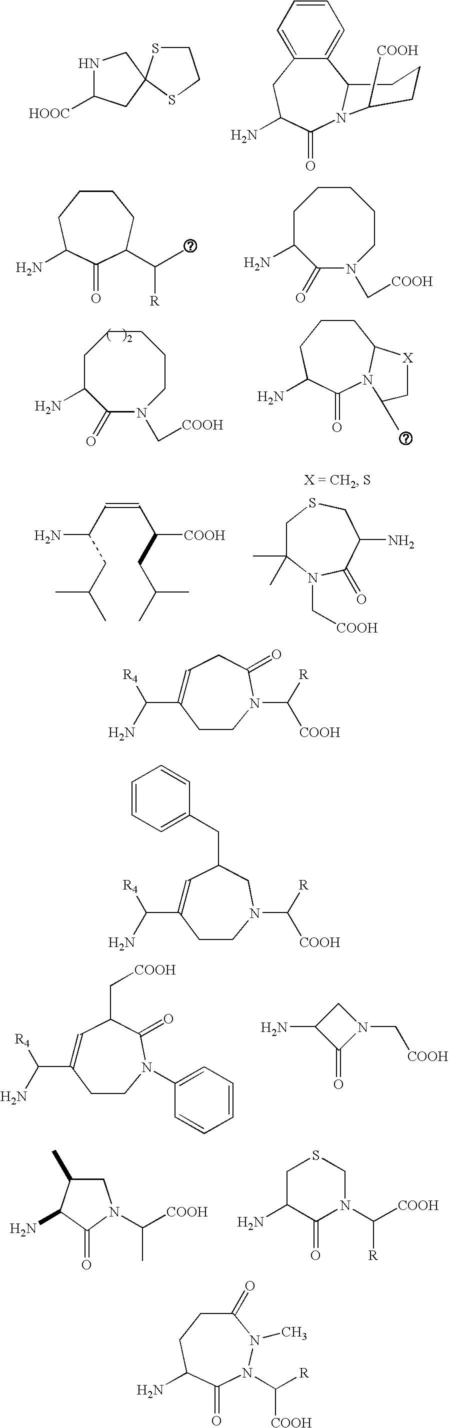

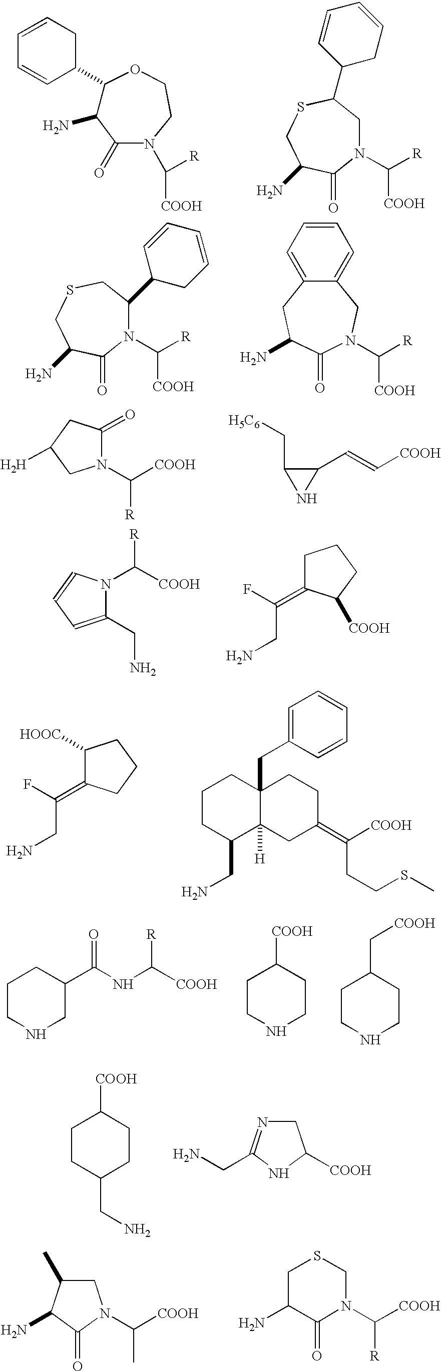

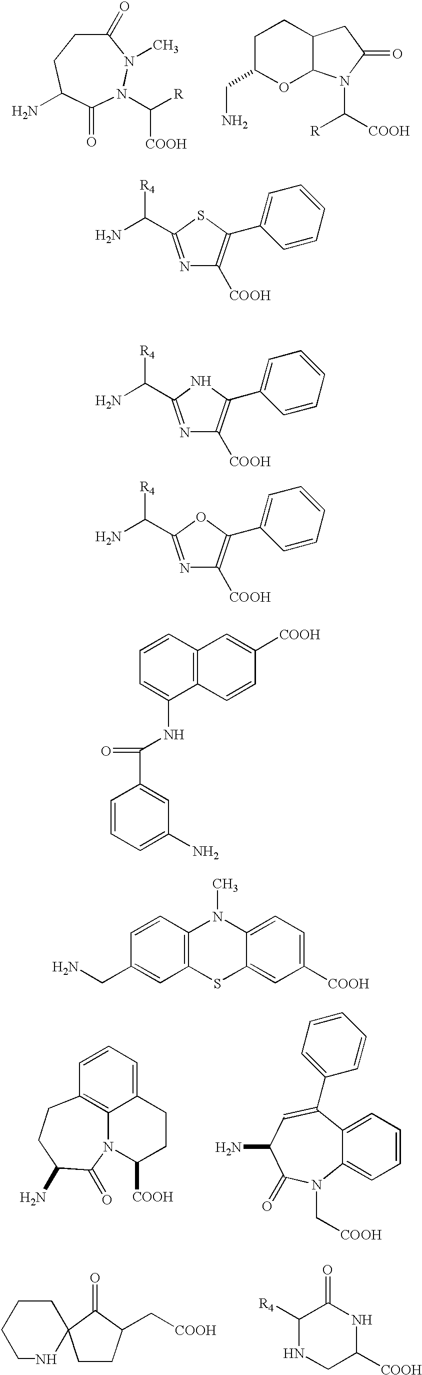

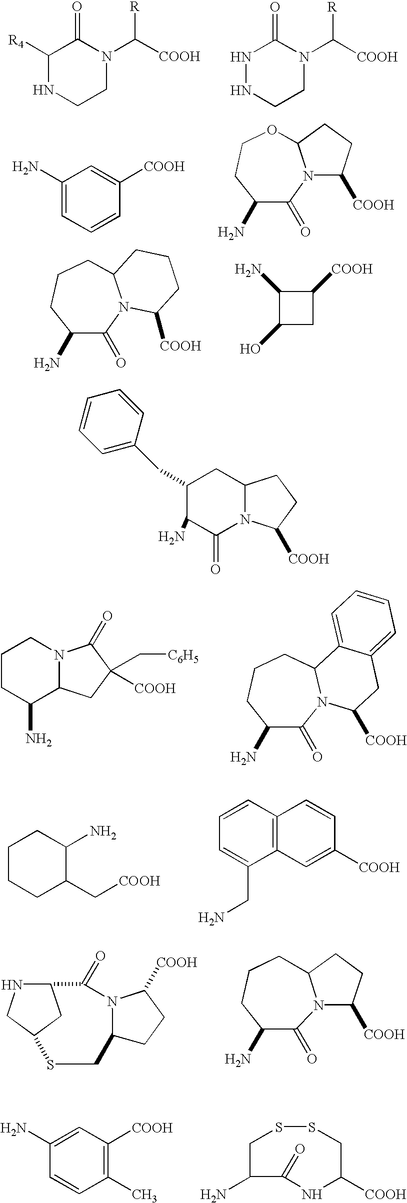

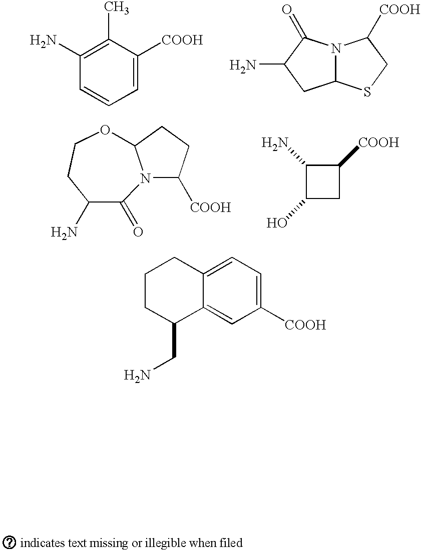

16. The method as claimed in claim 14, characterized in that:
a) a support resin carrying amino substituents is functionalized with a compound H—NR′-A-CO—OH (II′) which corresponds to the definition given for (II) and in which the amino group has been protected beforehand;
b) the fragment thus attached to the support resin is extended from the C-terminal side to the N-terminal side by (n−2) successive reactions for coupling the monomer (II), said monomer (II) being used in excess, various monomers (II) possibly being used in the successive coupling steps;
c) a final reaction is carried out for coupling a monomer protected on its N-terminal function with a group R₁ which is stable under the conditions under which the oligomer must be separated from the support;
d) the oligomer is separated from the support resin.

17. A method for preparing an artificial protein or an artificial polypeptide which is analogous to a natural protein or a natural polypeptide, consisting in carrying out solid-phase peptide synthesis coupling reactions, characterized in that, in the succession of reactions for coupling the α-amino acids constituting the natural polypeptide or protein, one or more α-amino acid sequences are replaced with an oligomer (I) or (I′), the length of which is equivalent to that of the α-amino acid sequence replaced.

18. An artificial polypeptide or protein which is analogous to a given natural polypeptide or protein, comprising one or more structuring fragments and one or more peptide fragments, characterized in that the peptide fragment(s) is (are) identical to those of the corresponding natural polypeptide or protein, and in that the structuring fragment(s) consist(s) of a fragment of an oligomer (I) or (I′), the length of which is substantially identical to that of the α-helical structuring component of the natural polypeptide or protein.

19. A protein analogous to hCRF (human corticotropin releasing factor), characterized in that it corresponds to one of the following formulae:
H-Ser-Glu-Glu-Pro-Pro-(DBT)₈-Lys-Leu-Met-Glu-Ile-Ile-NH₂
or
H-Ser-Glu-Glu-Pro-Pro-(DBT)₉-Leu-Met-Glu-Ile-Ile-NH₂
in which DBT is a fragment derived from (3S)-[amino]-5-(carbonylmethyl)-2,3-dihydro-1,5-benzothiazepin-4(5H)-one.

20. A protein analogous to hCRF (human corticotropin releasing factor), characterized in that it corresponds to the following formula:
H-Ser-Glu-Glu-Pro-Pro-(A₁)20-Arg-Lys-Leu-Met-Glu-Ile-Ile-NH₂
in which A₁ is a group derived from is the 3-(S)-amino-1-carbonylmethylpyrrolidin-2-one and represented by the formula [chemical structure]
